SEQUENCE LISTING (106 sequences)

SEQ ID NO: 1 - protein, 120 aa, Mus musculus
  REGION 1..120: note = Heavy Chain
QVQLQQPGAELVKPGASVKVSCKASGYTFTNYWMYWVKQRPGQGLEWIGRIHPSDSDTNYNQKFKGKATLTVDKSSSTAYMQLSSLTSEDSAVYYCAIEIYDGYNTMDYWGQGTSVTVSS

SEQ ID NO: 2 - protein, 120 aa, Mus musculus
  REGION 1..120: note = Heavy Chain
QVQLQQPGAELVKPGASVTVSCKSSGYTFTSYWVHWVKQRPGQGLEWIGRIHPSDSDTNYNQKFKGKATLTVDKSSTTAYIHLSSLTSEDSAVYYCAIEAITTVITTDYWGQGTTLTVSS

SEQ ID NO: 3 - protein, 119 aa, Mus musculus
  REGION 1..119: note = Heavy Chain
QVQLQQSGPELVKPGASVKISCKASGYTFTDYYINWVKQRPGQGLEWIGWIFPGSGSTYYNEKFKGKATLTVDKSSSTAYMLLSSLTSEDSAVYFCARRDYGSSYFDYWGQGTTLTVSS

SEQ ID NO: 4 - protein, 122 aa, Mus musculus
  REGION 1..122: note = Heavy Chain
QVQLQQSGPELVKPGASVKLSCKASGYTFTSYDINWVKQRPGQGLEWIGWIFPRDGTTKYNEKFKGKATLTVDTSSNTAYMEVHSLTSEDSAVYFCAIEGFTTVVARNFDVWGTGTTVTVSS

SEQ ID NO: 5 - protein, 116 aa, Mus musculus
  REGION 1..116: note = Heavy Chain
QVQLQQPGTDLVKPGASVKLSCKASGYTFTSYWMHWVKQRPGQGLEWIGNIDPNNGGTNQNEKFKNKATLTVDKSSSTAYMHLSSLTSEDSAVYYCAIEVGYYDYWGQGTTLTVSS

SEQ ID NO: 6 - protein, 120 aa, Mus musculus
  REGION 1..120: note = Heavy Chain
QVQLQQPGAELVKPGASVKVSCKASGYTFTSQWMYWVKQRPGQGLEWIGRIHPSDSDTNYNQKFKGKATLTVDKSSSTAYMQLSSLTSEDSAVYYCAIEIYDGYYAMDYWGQGTSVTVSS

SEQ ID NO: 7 - protein, 115 aa, Mus musculus
  REGION 1..115: note = Heavy Chain
QVQLQQPGAELVKPGASVKLSCKASGYTFTSYWMQWVKQRPGQGLEWIGEIDPSDSYTNYNQKFKGKATLTVDTSSSTAYMQLSSLTSEDSAVYYCARWLPFAYWGQGTLVTVSA

SEQ ID NO: 8 - protein, 114 aa, Mus musculus
  REGION 1..114: note = Heavy Chain
YMQLSSLTSEDSAVYYCAIEIYDGYYTMDYWGQGTSVTVSSAKTTPPSVYPLAPGSAAQTNSMVTLGCLVKGYFPEPVTVTWNSGSLSSGVHTFPAVLQSDLYTLSSSVTVPSS

SEQ ID NO: 9 - protein, 120 aa, Mus musculus
  REGION 1..120: note = Heavy Chain
QVQLQQPGAELVKPGASVKVSCKASGYTFTNYWMHWVKQRPGQGLEWIGRIHPSESDTNYNQKFKGKATLTVDNSSSTAYMQLSSLTSEDSAVYYCAMEDYYGSSYEDYWGQGTTLTVSS

SEQ ID NO: 10 - protein, 115 aa, Mus musculus
  REGION 1..115: note = Heavy Chain
QVQLKQSGPGLVQPSQSLSITCTVSGFSLTSYDVHWVRQSPGKGLEWLGVIWSGGSTDYNAAFISRLSIRKDNSKSQVFFKMNSLQADDTAIYYCATEVGYFDYWGQGTTLTVSS

SEQ ID NO: 11 - protein, 107 aa, Mus musculus
  REGION 1..107: note = Light Chain
DIVMTQSQKFMSTSVGDRVSVTCKASQNVGTNVAWYQQKPGQSPKALIYSASYRYSGVPDRFTGSGSGTDFTLTISNVQSEDLAEYFCQQYNNYPLTFGAGTKLELK

SEQ ID NO: 12 - protein, 107 aa, Mus musculus
  REGION 1..107: note = Light Chain
DIVMTQSQKFMSTSVGDRVSVTCKASQNVGTNVAWYQQKPGQSPKALIYSASYRYSGVPDRFTGSGSGTDFTLTISNVQSEDLAEFFCQQYNNFPYTFGGGTQLEMK

SEQ ID NO: 13 - protein, 112 aa, Mus musculus
  REGION 1..112: note = Light Chain
DVVLTQTPLSLPVNIGDQASISCKSTKSLLNSDGFTYLDWYLQKPGQSPQLLIYLVSNRFSGVPDRFSGSGSGTDFTLKISRVEAEDLGVYYCFQSNYLPLTFGSGTKLEIK

SEQ ID NO: 14 - protein, 107 aa, Mus musculus
  REGION 1..107: note = Light Chain
DIVMTQSQKFMSTSVGDRVSVTCKASQNVDTNVAWYQQKPGQSPKALIYSASYRYSGVPDRFTGSGSGTDFTLTISNVQSEDLADYFCQQYNTYPYTFGGGTKLEIK

SEQ ID NO: 15 - protein, 107 aa, Mus musculus
  REGION 1..107: note = Light Chain
DIVMTQSQKFMSTSVGDRVSVTCKASQNVGTNVVWYQQKPGQSPKALIYSASYRYSGVPDRFTGSGSGTDFTLTISNVQSEDLTEYFCEQYNSYPYTFGGGTKLEIK

SEQ ID NO: 16 - protein, 107 aa, Mus musculus
  REGION 1..107: note = Light Chain
DIVMTQSQKFMSTSVGDRVSVTCKASQNVGTNVAWYQQKPGQSPKALIYSASHRYSGVPDRFTGSGSGTDFTLTISNVQSEDLAEYFCQQYNSYPLTFGAGTKLELK

SEQ ID NO: 17 - protein, 86 aa, Mus musculus
  REGION 1..86: note = Light Chain
SLLNSSNQKNYLAWYQQKPGQSPKLLVYFASTRESGVPDRFIGSGSGTDFTLTISSVQAEDLADYFCQQHYSTPYTFGGGTKLEIK

SEQ ID NO: 18 - protein, 107 aa, Mus musculus
  REGION 1..107: note = Light Chain
DIVMTQSQKFMSTSVGDRISVSCKASQNVGTNVAWYQQKPGQSPKALIYSASYRYSGVPDRFTGSGSGTDFTLTISNVQSEDLAEYFCQQYNSYPLTFGAGTKLELK

SEQ ID NO: 19 - protein, 107 aa, Mus musculus
  REGION 1..107: note = Light Chain
DIVMTQSQKFMSTSVGDRVSVTCKASQNVGTNVAWYQQKPGQSPKALIYSASYRYSGVPDRFTGSGSGTDFTLTISDVQSEDLTEYFCEQYNSYPYTFGGGTKLEIK

SEQ ID NO: 20 - protein, 107 aa, Mus musculus
  REGION 1..107: note = Light Chain
DIVMTQSQKFMSTSVGDRVSVTCKASQNVGTNVAWYQQKPGQSPKALIYSASYRYSGVPDRFTGSGSGTDFTLTISNVQSEDLAEYFCQQYNTYPYTFGGGTKLEIK

SEQ ID NO: 21 - protein, 107 aa, Mus musculus
  REGION 1..107: note = Light Chain
DIVMTQSQKFMSTSVGDRVSVTCKASQNVGTNVAWYQQKPGQSPKALIYSASYRYSGVPDRFTGSGSGTDFSLTISNVQSEDLAEYFCQQYNSYPYTFGGGTKLEIK

SEQ ID NO: 22 - protein, 25 aa, synthetic construct
  REGION 1..25: note = FR domain
QVQLQQPGAELVKPGASVKVSCKAS

SEQ ID NO: 23 - protein, 25 aa, synthetic construct
  REGION 1..25: note = FR domain
QVQLQQPGAELVKPGASVTVSCKSS

SEQ ID NO: 24 - protein, 25 aa, synthetic construct
  REGION 1..25: note = FR domain
QVQLQQSGPELVKPGASVKISCKAS

SEQ ID NO: 25 - protein, 25 aa, synthetic construct
  REGION 1..25: note = FR domain
QVQLQQSGPELVKPGASVKLSCKAS

SEQ ID NO: 26 - protein, 25 aa, synthetic construct
  REGION 1..25: note = FR domain
QVQLQQPGTDLVKPGASVKLSCKAS

SEQ ID NO: 27 - protein, 25 aa, synthetic construct
  REGION 1..25: note = FR domain
QVQLQQPGAELVKPGASVKLSCKAS

SEQ ID NO: 28 - protein, 25 aa, synthetic construct
  REGION 1..25: note = FR domain
QVQLKQSGPGLVQPSQSLSITCTVS

SEQ ID NO: 29 - protein, 26 aa, synthetic construct
  REGION 1..26: note = FR domain
DIVMTQSQKFMSTSVGDRVSVTCKAS

SEQ ID NO: 30 - protein, 26 aa, synthetic construct
  REGION 1..26: note = FR domain
DVVLTQTPLSLPVNIGDQASISCKST

SEQ ID NO: 31 - protein, 26 aa, synthetic construct
  REGION 1..26: note = FR domain
DIVMTQSQKFMSTSVGDRISVSCKAS

SEQ ID NO: 32 - protein, 7 aa, synthetic construct
  REGION 1..7: note = CDR domain
GYTFTNY

SEQ ID NO: 33 - protein, 7 aa, synthetic construct
  REGION 1..7: note = CDR domain
GYTFTSY

SEQ ID NO: 34 - protein, 7 aa, synthetic construct
  REGION 1..7: note = CDR domain
GYTFTDY

SEQ ID NO: 35 - protein, 8 aa, synthetic construct
  REGION 1..8: note = CDR domain
GYTFTSYD

SEQ ID NO: 36 - protein, 7 aa, synthetic construct
  REGION 1..7: note = CDR domain
GYTFTSQ

SEQ ID NO: 37 - protein, 7 aa, synthetic construct
  REGION 1..7: note = CDR domain
GFSLTSY

SEQ ID NO: 38 - protein, 6 aa, synthetic construct
  REGION 1..6: note = CDR domain
QNVGTN

SEQ ID NO: 39 - protein, 11 aa, synthetic construct
  REGION 1..11: note = CDR domain
KSLLNSDGFTY

SEQ ID NO: 40 - protein, 6 aa, synthetic construct
  REGION 1..6: note = CDR domain
QNVDTN

SEQ ID NO: 41 - protein, 11 aa, synthetic construct
  REGION 1..11: note = CDR domain
SLLNSSNQKNY

SEQ ID NO: 42 - protein, 18 aa, synthetic construct
  REGION 1..18: note = FR domain
WMYWVKQRPGQGLEWIGR

SEQ ID NO: 43 - protein, 18 aa, synthetic construct
  REGION 1..18: note = FR domain
WVHWVKQRPGQGLEWIGR

SEQ ID NO: 44 - protein, 18 aa, synthetic construct
  REGION 1..18: note = FR domain
YINWVKQRPGQGLEWIGW

SEQ ID NO: 45 - protein, 18 aa, synthetic construct
  REGION 1..18: note = FR domain
WMHWVKQRPGQGLEWIGN

SEQ ID NO: 46 - protein, 18 aa, synthetic construct
  REGION 1..18: note = FR domain
WMQWVKQRPGQGLEWIGE

SEQ ID NO: 47 - protein, 18 aa, synthetic construct
  REGION 1..18: note = FR domain
WMHWVKQRPGQGLEWIGR

SEQ ID NO: 48 - protein, 18 aa, synthetic construct
  REGION 1..18: note = FR domain
DVHWVRQSPGKGLEWLGV

SEQ ID NO: 49 - protein, 17 aa, synthetic construct
  REGION 1..17: note = FR domain
VAWYQQKPGQSPKALIY

SEQ ID NO: 50 - protein, 17 aa, synthetic construct
  REGION 1..17: note = FR domain
LDWYLQKPGQSPQLLIY

SEQ ID NO: 51 - protein, 17 aa, synthetic construct
  REGION 1..17: note = FR domain
VVWYQQKPGQSPKALIY

SEQ ID NO: 52 - protein, 17 aa, synthetic construct
  REGION 1..17: note = FR domain
LAWYQQKPGQSPKLLVY

SEQ ID NO: 53 - protein, 8 aa, synthetic construct
  REGION 1..8: note = CDR domain
IHPSDSDT

SEQ ID NO: 54 - protein, 8 aa, synthetic construct
  REGION 1..8: note = CDR domain
IFPGSGST

SEQ ID NO: 55 - skipped

SEQ ID NO: 56 - protein, 8 aa, synthetic construct
  REGION 1..8: note = CDR domain
IDPNNGGT

SEQ ID NO: 57 - protein, 8 aa, synthetic construct
  REGION 1..8: note = CDR domain
IDPSDSYT

SEQ ID NO: 58 - protein, 8 aa, synthetic construct
  REGION 1..8: note = CDR domain
IHPSESDT

SEQ ID NO: 59 - protein, 7 aa, synthetic construct
  REGION 1..7: note = CDR domain
IWSGGST

SEQ ID NO: 60-62 - skipped (3 entries)

SEQ ID NO: 63 - protein, 38 aa, synthetic construct
  REGION 1..38: note = FR domain
NYNQKFKGKATLTVDKSSSTAYMQLSSLTSEDSAVYYC

SEQ ID NO: 64 - protein, 38 aa, synthetic construct
  REGION 1..38: note = FR domain
NYNQKFKGKATLTVDKSSTTAYIHLSSLTSEDSAVYYC

SEQ ID NO: 65 - protein, 38 aa, synthetic construct
  REGION 1..38: note = FR domain
YYNEKFKGKATLTVDKSSSTAYMLLSSLTSEDSAVYFC

SEQ ID NO: 66 - protein, 38 aa, synthetic construct
  REGION 1..38: note = FR domain
KYNEKFKGKATLTVDTSSNTAYMEVHSLTSEDSAVYFC

SEQ ID NO: 67 - protein, 38 aa, synthetic construct
  REGION 1..38: note = FR domain
NQNEKFKNKATLTVDKSSSTAYMHLSSLTSEDSAVYYC

SEQ ID NO: 68 - protein, 38 aa, synthetic construct
  REGION 1..38: note = FR domain
NYNQKFKGKATLTVDTSSSTAYMQLSSLTSEDSAVYYC

SEQ ID NO: 69 - protein, 17 aa, synthetic construct
  REGION 1..17: note = FR domain
YMQLSSLTSEDSAVYYC

SEQ ID NO: 70 - protein, 38 aa, synthetic construct
  REGION 1..38: note = FR domain
NYNQKFKGKATLTVDNSSSTAYMQLSSLTSEDSAVYYC

SEQ ID NO: 71 - protein, 38 aa, synthetic construct
  REGION 1..38: note = FR domain
DYNAAFISRLSIRKDNSKSQVFFKMNSLQADDTAIYYC

SEQ ID NO: 72 - protein, 36 aa, synthetic construct
  REGION 1..36: note = FR domain
YRYSGVPDRFTGSGSGTDFTLTISNVQSEDLAEYFC

SEQ ID NO: 73 - protein, 36 aa, synthetic construct
  REGION 1..36: note = FR domain
YRYSGVPDRFTGSGSGTDFTLTISNVQSEDLAEFFC

SEQ ID NO: 74 - protein, 36 aa, synthetic construct
  REGION 1..36: note = FR domain
NRFSGVPDRFSGSGSGTDFTLKISRVEAEDLGVYYC

SEQ ID NO: 75 - protein, 36 aa, synthetic construct
  REGION 1..36: note = FR domain
YRYSGVPDRFTGSGSGTDFTLTISNVQSEDLADYFC

SEQ ID NO: 76 - protein, 36 aa, synthetic construct
  REGION 1..36: note = FR domain
YRYSGVPDRFTGSGSGTDFTLTISNVQSEDLTEYFC

SEQ ID NO: 77 - protein, 36 aa, synthetic construct
  REGION 1..36: note = FR domain
HRYSGVPDRFTGSGSGTDFTLTISNVQSEDLAEYFC

SEQ ID NO: 78 - protein, 36 aa, synthetic construct
  REGION 1..36: note = FR domain
TRESGVPDRFIGSGSGTDFTLTISSVQAEDLADYFC

SEQ ID NO: 79 - protein, 36 aa, synthetic construct
  REGION 1..36: note = FR domain
YRYSGVPDRFTGSGSGTDFTLTISDVQSEDLTEYFC

SEQ ID NO: 80 - protein, 36 aa, synthetic construct
  REGION 1..36: note = FR domain
YRYSGVPDRFTGSGSGTDFSLTISNVQSEDLAEYFC

SEQ ID NO: 81 - protein, 13 aa, synthetic construct
  REGION 1..13: note = CDR domain
AIEIYDGYNTMDY

SEQ ID NO: 82 - protein, 13 aa, synthetic construct
  REGION 1..13: note = CDR domain
AIEAITTVITTDY

SEQ ID NO: 83 - protein, 12 aa, synthetic construct
  REGION 1..12: note = CDR domain
ARRDYGSSYFDY

SEQ ID NO: 84 - protein, 15 aa, synthetic construct
  REGION 1..15: note = CDR domain
AIEGFTTVVARNFDV

SEQ ID NO: 85 - protein, 9 aa, synthetic construct
  REGION 1..9: note = CDR domain
AIEVGYYDY

SEQ ID NO: 86 - protein, 13 aa, synthetic construct
  REGION 1..13: note = CDR domain
AIEIYDGYYAMDY

SEQ ID NO: 87 - protein, 8 aa, synthetic construct
  REGION 1..8: note = CDR domain
ARWLPFAY

SEQ ID NO: 88 - protein, 13 aa, synthetic construct
  REGION 1..13: note = CDR domain
AIEIYDGYYTMDY

SEQ ID NO: 89 - protein, 13 aa, synthetic construct
  REGION 1..13: note = CDR domain
AMEDYYGSSYEDY

SEQ ID NO: 90 - protein, 9 aa, synthetic construct
  REGION 1..9: note = CDR domain
ATEVGYFDY

SEQ ID NO: 91 - protein, 9 aa, synthetic construct
  REGION 1..9: note = CDR domain
QQYNNYPLT

SEQ ID NO: 92 - protein, 9 aa, synthetic construct
  REGION 1..9: note = CDR domain
QQYNNFPYT

SEQ ID NO: 93 - protein, 9 aa, synthetic construct
  REGION 1..9: note = CDR domain
FQSNYLPLT

SEQ ID NO: 94 - protein, 9 aa, synthetic construct
  REGION 1..9: note = CDR domain
QQYNTYPYT

SEQ ID NO: 95 - protein, 9 aa, synthetic construct
  REGION 1..9: note = CDR domain
EQYNSYPYT

SEQ ID NO: 96 - protein, 9 aa, synthetic construct
  REGION 1..9: note = CDR domain
QQYNSYPLT

SEQ ID NO: 97 - protein, 9 aa, synthetic construct
  REGION 1..9: note = CDR domain
QQHYSTPYT

SEQ ID NO: 98 - skipped

SEQ ID NO: 99 - protein, 11 aa, synthetic construct
  REGION 1..11: note = FR domain
WGQGTSVTVSS

SEQ ID NO: 100 - protein, 11 aa, synthetic construct
  REGION 1..11: note = FR domain
WGQGTTLTVSS

SEQ ID NO: 101 - protein, 11 aa, synthetic construct
  REGION 1..11: note = FR domain
WGTGTTVTVSS

SEQ ID NO: 102 - protein, 11 aa, synthetic construct
  REGION 1..11: note = FR domain
WGQGTLVTVSA

SEQ ID NO: 103 - protein, 10 aa, synthetic construct
  REGION 1..10: note = FR domain
FGAGTKLELK

SEQ ID NO: 104 - protein, 10 aa, synthetic construct
  REGION 1..10: note = FR domain
FGGGTQLEMK

SEQ ID NO: 105 - protein, 10 aa, synthetic construct
  REGION 1..10: note = FR domain
FGSGTKLEIK

SEQ ID NO: 106 - protein, 10 aa, synthetic construct
  REGION 1..10: note = FR domain
FGGGTKLEIK

(19) United States
(12) Patent Application Publication
(10) Pub. No.: US 2024/0067755 A1
(43) Pub. Date: Feb. 29, 2024

(54) ANTI-FENTANYL ANTIBODIES

(71) Applicants: Deutsches Krebsforschungszentrum Stiftung des öffentlichen Rechts, Heidelberg (DE); Hepione Therapeutics Inc., New York, NY (US)

(72) Inventors: Nina Papavasiliou, Heidelberg (DE); Johanna Triller, Heidelberg (DE); Paraskevi Vlachou-Efstathiou, Heidelberg (DE); Erec Stebbins, Heidelberg (DE); Monique van Straaten, Heidelberg (DE); Johann Zeelen, Heidelberg (DE); Joseph Verdi, New York, NY (US); Yosip Kelemen, New York, NY (US)

(21) Appl. No.: 17/823,025

(22) Filed: Aug. 29, 2022

(51) Int. Cl. C07K 16/44

(52) U.S. Cl. CPC C07K 16/44; A61K 2039/505; C07K 2317/92

(57) ABSTRACT

The present invention concerns the field of antibodies. More specifically, it relates to an antibody which specifically binds to a hapten being fentanyl or a derivative thereof, said antibody binding to the hapten with an equilibrium dissociation constant (Kd) of at most 1.000 pM, at most 800 pM, at most 600 pM, at most 400 pM, at most 200 pM, at most 100 pM or at most 75 pM, wherein the binding pocket for the hapten comprises amino acids from all three complementary determining regions (CDRs) of each chain. The present invention also relates to a polynucleotide encoding said antibody, a vector or expression construct comprising the polynucleotide, a host cell comprising the polynucleotide or the vector or expression construct or a non-human transgenic organism comprising said polynucleotide or said vector or expression construct. The invention also relates to said antibody or said polynucleotide for use as a medicament for treating and/or preventing a disease or condition in a subject associated with administration fentanyl or a derivative thereof.

### SEQUENCE LISTING

This application incorporates by reference the material in the ST.26 XML file titled DK16402_US_Sequence_listing_ASD05-55, which was created on Aug. 29, 2022 and is 126 KB.

The present invention concerns the field of antibodies. More specifically, it relates to an antibody which specifically binds to a hapten being fentanyl or a derivative thereof, said antibody binding to the hapten with an equilibrium dissociation constant (Kd) of at most 1.000 pM, at most 800 pM, at most 600 pM, at most 400 pM, at most 200 pM, at most 100 pM or at most 75 pM, wherein the binding pocket for the hapten comprises amino acids from all three complementary determining regions (CDRs) of each chain and wherein said antibody is capable of protecting mice from adverse fentanyl actions when administered at a dosage (antibody compound (mg)/mouse body weight (kg)) of at most 10 mg/kg, at most 8 mg/kg, at most 6 mg/kg, at most 5 mg/kg, at most 4 mg/kg at most 3 mg/kg at most 2 mg/kg or at most 1 mg/kg. The present invention also relates to a polynucleotide encoding said antibody, a vector or expression construct comprising the polynucleotide, a host cell comprising the polynucleotide or the vector or expression construct or a non-human transgenic organism comprising said polynucleotide or said vector or expression construct. The invention also relates to said antibody or said polynucleotide for use as a medicament for treating and/or preventing a disease or condition in a subject associated with administration fentanyl or a derivative thereof.

Fentanyl is a synthetic opioid in the phenylpiperidine family, which includes sufentanil, alfentanil, remifentanil, carfentanil, acetylfentanil and norfentanil used as a pain therapeutic and for anesthesia.

The duration of action of fentanyl has been underestimated leading to harm in a medical context as described elsewhere herein in more detail. However, besides its therapeutic use, fentanyl is frequently abused as an illegal recreational narcotic drug and as such also causes harm for the individual and the society. It is more potent than heroin and its abuse has resulted in many deaths, in particular in abusers lacking opioid tolerance.

Fentanyl acts via opioid receptors. These receptors are G-protein-coupled seven transmembrane receptors. The extracellular N-terminus is important in differentiating different types of binding substrates. When fentanyl binds, downstream signaling leads to the inhibitory effects, such as decreased cAMP production, decreased calcium ion influx, and increased potassium efflux. This inhibits the ascending pathways in the central nervous system to increase pain threshold by changing the perception of pain mediated by decreasing propagation of nociceptive signals, resulting in analgesic effects.

As a μ-receptor agonist, fentanyl binds 50 to 100 times more potently than morphine. It can also bind to the delta and kappa opioid receptors but with a lower affinity. It has high lipid solubility, allowing it to more easily enter into the central nervous system. Fentanyl can produce the following clinical effects strongly, through agonistic action on μ-receptors: supraspinal analgesia (μl), respiratory depression (μ2), physical dependence, and muscle rigidity. Moreover, it produces sedation and spinal analgesia through K-receptor agonism.

The following therapeutic effects are known for fentanyl: pain relief, primarily, fentanyl provides the relief of pain by acting on the brain and spinal μ-receptors; sedation, fentanyl produces sleep and drowsiness, as the dosage is increased, and can produce the δ-waves often seen in natural sleep on electroencephalogram; suppression of the cough reflex, fentanyl can decrease the struggle against an endotracheal tube and excessive coughing by decreasing the cough reflex, becoming useful when intubating people who are awake and have compromised airways. After receiving a bolus dose of fentanyl, people can also experience paradoxical coughing, which is a phenomenon that is not well understood.

Moreover, there are various side effects known for fentanyl. Its most common side effects, typically affecting more than 10% of people receiving it, include nausea, vomiting, constipation, dry mouth, somnolence, confusion, and asthenia (weakness). Less frequently, in 3 to 10% of people, fentanyl can cause abdominal pain, headache, fatigue, anorexia and weight loss, dizziness, nervousness, anxiety, depression, flu-like symptoms, dyspepsia (indigestion), shortness of breath, hypoventilation, apnoea, and urinary retention. Fentanyl use has also been associated with aphasia.

There are functional and/or structural derivatives of fentanyl available, for example, the fentanyl analogues alfentanil, sufentanil, remifentanil, carfentanil, acetylfentanil and norfentanil. The aforementioned fentanyl derivatives are also used for therapeutic purposes in human and veterinary medicine. Nevertheless, there is also abuse as narcotic drugs. Moreover, carfentanil and remifentanil have been reported to be abused as weapon in the form of aerosol mist.

In a clinical environment, overdosing of fentanyl can be treated by administration of antagonistically acting naloxone. For the treatment of addiction caused by abuse of fentanyl or its derivatives, behavioral therapy together with the administration of therapeutics such as buprenorphine, methadone or naltrexone is used.

There have been reports of monoclonal antibodies that based on mice models could be used to treat overdosing of fentanyl or carfentanil opioid abuse disorders (Smith 2019). The antibodies reported confer a partial protection, however, at rather high dosages, only.

However, there is a need for efficient antagonistically acting compounds for treating overdosing, side effects, toxic effects and abuse of fentanyl or its derivatives.

The technical problem underlying the present invention may be seen as the provision of means and methods for complying with the aforementioned needs. The technical problem is solved by the embodiments characterized in the claims and herein below.

Thus, the present invention relates to an antibody which specifically binds to a hapten being fentanyl or a derivative thereof, said antibody binding to the hapten with an equilibrium dissociation constant (Kd) of at most 1.000 pM, at most 800 pM, at most 600 pM, at most 400 pM, at most 200 pM, at most 100 pM or at most 75 pM, wherein the binding pocket for the hapten comprises amino acids from all three complementary determining regions (CDRs) of each chain and wherein said antibody is capable of protecting mice from adverse fentanyl actions when administered at a dosage (antibody compound (mg)/mouse body weight (kg)) of at most 10 mg/kg, at most 8 mg/kg, at most 6 mg/kg, at most 5 mg/kg, at most 4 mg/kg at most 3 mg/kg at most 2 mg/kg or at most 1 mg/kg.

It is to be understood that in the specification and in the claims, “a” or “an” can mean one or more of the items referred to in the following depending upon the context in which it is used. Thus, for example, reference to “an” item can mean that at least one item can be utilized.

As used in the following, the terms “have”, “comprise” or “include” are meant to have a non-limiting meaning or a limiting meaning. Thus, having a limiting meaning these terms may refer to a situation in which, besides the feature introduced by these terms, no other features are present in an embodiment described, i.e. the terms have a limiting meaning in the sense of “consisting of” or “essentially consisting of”. Having a non-limiting meaning, the terms refer to a situation where besides the feature introduced by these terms, one or more other features are present in an embodiment described.

Further, as used in the following, the terms “preferably”, “more preferably”, “most preferably”, “particularly”, “more particularly”, “typically”, and “more typically” are used in conjunction with features in order to indicate that these features are preferred features, i.e. the terms shall indicate that alternative features may also be envisaged in accordance with the invention.

Further, it will be understood that the term “at least one” as used herein means that one or more of the items referred to following the term may be used in accordance with the invention. For example, if the term indicates that at least one item shall be used this may be understood as one item or more than one item, i.e. two, three, four, five or any other number. Depending on the item the term refers to the skilled person understands as to what upper limit the term may refer, if any.

The term “antibody” as used herein refers to any immunoglobulin polypeptide derived from VDJ genomic sequences which comprises amino acid sequence stretches that are capable of forming a binding pocket that is sufficient for specific hapten binding with an equilibrium dissociation constant (Kd) as referred to herein. Such an antibody may be, preferably, a monoclonal antibody, a single chain antibody, a chimeric antibody or any fragment or derivative of such antibodies being still capable of binding to the hapten specifically as referred to herein. Fragments and derivatives comprised by the term antibody as used herein encompass a bispecific antibody, a synthetic antibody, a Fab, F(ab)2, Fv or scFv fragment or a chemically modified derivative of any of these antibodies. Antibodies or fragments thereof, in general, can be obtained by using methods which are described, e.g., in Harlow and Lane “Antibodies, A Laboratory Manual”, CSH Press, Cold Spring Harbor, 1988. Monoclonal antibodies can be prepared by the techniques which comprise the fusion of mouse myeloma cells to spleen cells derived from immunized mammals and, preferably, immunized mice. The antibody of the present invention can be, preferably, generated by using the techniques described in the accompanying Examples below.

The antibody according to the invention shall comprise three complementary determining regions in each chain. The term “complementary determining region (CDR)” as used herein refers to regions in the variable domains of the heavy and light chain of an antibody that define the binding affinity and specificity of the antibody. There are three CDRs for the heavy chain, CDR1-H, CDR2-H and CDR3-H, and three CDRs for the light chain, CDR1-L, CDR2-L, and CDR3-L.

Preferably, the antibody of the invention comprises at least one heavy chain CDR, said heavy chain CDR being one of the heavy chain CDRs specified as follows:

Preferably, the antibody of the present invention comprises a heavy chain CDR1 having an amino acid sequence selected from the group consisting of:
(a) an amino acid sequence as shown in any one of SEQ ID NOs: 32, 33, 34, 35, 36, or 37; and
(b) an amino acid sequence which differs by at least one amino acid exchange, deletion and/or addition from the sequence as shown in any one of SEQ ID NOs: 32, 33, 34, 35, 36, or 37.

Also preferably, the antibody of the present invention comprises a heavy chain CDR2 having an amino acid sequence selected from the group consisting of:
(a) an amino acid sequence as shown in any one of SEQ ID NOs: 53, 54, 55, 56, 57, 58, or 59; and
(b) an amino acid sequence which differs by at least one amino acid exchange, deletion and/or addition from the sequence as shown in any one of SEQ ID NOs: 53, 54, 56, 57, 58, or 59.

Further preferably, the antibody of the present invention comprises a heavy chain CDR3 having an amino acid sequence selected from the group consisting of:
(a) an amino acid sequence as shown in any one of SEQ ID NOs: 81, 82, 83, 84, 85, 86, 87, 89, or 90; and
(b) an amino acid sequence which differs by at least one amino acid exchange, deletion and/or addition from the sequence as shown in any one of SEQ ID NOs: 81, 82, 83, 84, 85, 86, 87, 89, or 90.

More preferably, the antibody of the invention comprises a heavy chain CDR1, CDR2 and CDR3 selected from the aforementioned heavy chain CDRs.

Preferably, the antibody of the invention comprises at least one light chain CDR, said light chain CDR being one of the light chain CDRs specified as follows:

Preferably, the antibody of the present invention comprises a light chain CDR1 having an amino acid sequence selected from the group consisting of:
(a) an amino acid sequence as shown in any one of SEQ ID NOs: 38, 39, 40, or 41; and
(b) an amino acid sequence which differs by at least one amino acid exchange, deletion and/or addition from the sequence as shown in any one of SEQ ID NOs: 38, 39, 40, or 41.

Also preferably, the antibody of the present invention comprises a light chain CDR2 having an amino acid sequence selected from the group consisting of:
(a) an amino acid sequence as shown in any one of SEQ ID NOs: 60, 61, or 62; and
(b) an amino acid sequence which differs by at least one amino acid exchange, deletion and/or addition from the sequence as shown in any one of SEQ ID NOs: 60, 61, or 62.

Further preferably, the antibody of the present invention comprises a light chain CDR3 having an amino acid sequence selected from the group consisting of:
(a) an amino acid sequence as shown in any one of SEQ ID NOs: 91, 92, 93, 94, 95, 96, or 97; and
(b) an amino acid sequence which differs by at least one amino acid exchange, deletion and/or addition from the sequence as shown in any one of SEQ ID NOs: 91, 92, 93, 94, 95, 96, or 97.

More preferably, the antibody of the invention comprises a light chain CDR1, CDR2 and CDR3 selected from the aforementioned light chain CDRs.

It will be understood that a variant amino acid sequence which differs by at least one amino acid exchange, deletion and/or addition from any of the aforementioned amino acid sequences shall still be capable of exhibiting essentially the same immunological properties as the concrete amino acid sequence identified by a SEQ ID number.

More preferably, such a variant amino acid sequence is at least 70%, at least 75%, at least 80%, at least 85%, at least 90%, at least 95%, at least 96%, at least 97%, at least 98%, at least 99% identical to the concrete amino acid sequence identified by a SEQ ID No. Sequence identity between two amino acid sequences as referred to herein, in general, can be determined by alignment of two sequences either over the entire length of one of the sequences or within a comparison window. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment and calculation of sequence identity can be done by using published techniques or methods codified in computer programs such as, for example, BLASTP, BLASTN or FASTA. The percent sequence identity values are, preferably, calculated over the entire amino acid sequence. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp or the programs Gap and BestFit, which are part of the GCG software packet (Genetics Computer Group, US), may be used. The sequence identity values recited above in percent (%) are to be determined, in another aspect of the invention, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

In the case of the aforementioned variants of CDR sequences it is, however, preferably envisaged that the CDR amino acid sequences which differs by at least one amino acid exchange, deletion and/or addition differs from the specific sequence shown in any one of the CDR SEQ ID numbers by at most 3, at most 2 or at most 1 amino acid. Said at most 3, at most 2 or at most 1 amino acid may be deleted exchange or added.

More preferably, the antibody of the invention comprises in its heavy chain a combination of CDRs selected from the group consisting of:
SEQ ID NOs: 32, 53, and 81;
SEQ ID NOs: 33, 53, and 82;
SEQ ID NOs: 34, 54, and 83;
SEQ ID NOs: 35, 54, and 84;
SEQ ID NOs: 33, 56, and 85;
SEQ ID NOs: 36, 53, and 86;
SEQ ID NOs: 33, 57, and 87;
SEQ ID NOs: 33, 56, and 85;
SEQ ID NOs: 32, 58, and 89; and
SEQ ID NOs: 37, 59 and 90.

More preferably, the antibody of the invention comprises in its light chain a combination of CDRs selected from the group consisting of:
SEQ ID NOs: 38, 60, and 91;
SEQ ID NOs: 38, 60, and 92;
SEQ ID NOs: 39, 61, and 93;
SEQ ID NOs: 40, 60, and 94;
SEQ ID NOs: 38, 60, and 95;
SEQ ID NOs: 38, 60, and 96;
SEQ ID NOs: 41, 62, and 97;
SEQ ID NOs: 38, 60, and 94; and
SEQ ID NOs: 38, 60 and 97.

The three CDRs of the heavy and the three CDRS of the light chain of the antibody of the invention shall form a binding pocket for the hapten to be bound. The term “binding pocket” in accordance with the present invention refers to a three dimensional structure of the antibody of the invention required for hapten binding. The binding pocket comprises an arrangement of amino acids the side chains of which are capable of interacting by physico-chemical forces, such as Van-der-Waals interactions, hydrogen bonds, Pi-anion, Pi-Pi T-shaped or Pi-alkyl, with the hapten. The binding pocket of the antibody of the present invention is composed of amino acids from all three complementary determining regions (CDRs) of each chain. In addition, there may be additional amino acids from framework regions of the heavy and light chain that participate in forming the binding pocket. Moreover, preferred hapten binding pockets of the antibody of the present invention are shown in FIGS. 10A-10D. The amino acids which are, preferably, involved in the hapten binding pocket are indicated in said FIGS. 10A-10D. Thus, more preferably, the hapten binding pocket comprises at least the following amino acids, preferably, as shown in FIGS. 10A-10D:
(i) Trp 47, Tyr 35, Leu 96, Trp 110, Tyr 36, Ile 98, Tyr 91, Thr 106, Asp 108, Tyr 55, Tyr 49, Tyr 101, Gln 89, and Glu 99;
(ii) Val 37, His 35, Tyr 6, Ala 97, Tyr 96, Phe 98, Asp 104, Ile 98, Tyr 55, Tyr 102, Glu 98, Glu 99, Gly 101, Tyr 91, and Tyr 49
(iii) Val 37, Ala 97, His 35, asp 108, Phe 98, Met 98, Tyr 36, Tyr 96, Glu 99, Tyr 55, Tyr 91, Tyr 106, and Gln 89; or
(iv) Trp 110, Phe 98, Ala 97, His 35, Tyr 55, Ile 98, Tyr 96, Tyr 49, Asp 108, Tyr 91, Asn 32, Gln 89, and Glu 99.

Depending on the antibody type envisaged, the antibody of the invention may further comprise amino acids or amino acid sequence from the framework regions. The term “framework regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. From N-terminal to C-terminal, light chain variable region and heavy chain variable region both typically have the following order of these elements: FR1, CDR1, FR2, CDR2, FR3, CDR3 and FR4.

Preferably, the antibody according to the present invention also comprises a heavy chain FR1 having an amino acid sequence selected from the group consisting of:
(a) an amino acid sequence as shown in any one of SEQ ID NOs: 22, 23, 24, 25, 26, 27 or 28; and
(b) an amino acid sequence which differs by at least one amino acid exchange, deletion and/or addition from the sequence as shown in any one of SEQ ID NOs: 22, 23, 24, 25, 26, 27 or 28.

Preferably, the antibody according to the present invention comprises a heavy chain FR2 having an amino acid sequence selected from the group consisting of:
(a) an amino acid sequence as shown in any one of SEQ ID NOs: 42, 43, 44, 45, 46, 47 or 48; and
(b) an amino acid sequence which differs by at least one amino acid exchange, deletion and/or addition from the sequence as shown in any one of SEQ ID NOs: 42, 43, 44, 45, 46, 47 or 48.

Preferably, the antibody according to the present invention comprises a heavy chain FR3 having an amino acid sequence selected from the group consisting of:
(a) an amino acid sequence as shown in any one of SEQ ID NOs: 63, 64, 65, 66, 67, 68, 69, 70 or 71; and
(b) an amino acid sequence which differs by at least one amino acid exchange, deletion and/or addition from the sequence as shown in any one of SEQ ID NOs: 63, 64, 65, 66, 67, 68, 69, 70 or 71.

Preferably, the antibody according to the present invention comprises a heavy chain FR4 having an amino acid sequence selected from the group consisting of:
(a) an amino acid sequence as shown in any one of SEQ ID NOs: 99, 100, 101 or 102; and
(b) an amino acid sequence which differs by at least one amino acid exchange, deletion and/or addition from the sequence as shown in any one of SEQ ID NOs: 99, 100, 101 or 102.

Preferably, the antibody according to the present invention comprises a light chain FR1 having an amino acid sequence selected from the group consisting of:
(a) an amino acid sequence as shown in any one of SEQ ID NOs: 29, 30 or 31; and
(b) an amino acid sequence which differs by at least one amino acid exchange, deletion and/or addition from the sequence as shown in any one of SEQ ID NOs: 29, 30 or 31.

Preferably, the antibody according to the present invention comprises a light chain FR2 having an amino acid sequence selected from the group consisting of:
(a) an amino acid sequence as shown in any one of SEQ ID NOs: 49, 50, 51 or 52; and
(b) an amino acid sequence which differs by at least one amino acid exchange, deletion and/or addition from the sequence as shown in any one of SEQ ID NOs: 49, 50, 51 or 52.

Preferably, the antibody according to the present invention comprises a light chain FR3 having an amino acid sequence selected from the group consisting of:
(a) an amino acid sequence as shown in any one of SEQ ID NOs: 72, 73, 74, 75, 76, 77, 78, 79 or 80; and
(b) an amino acid sequence which differs by at least one amino acid exchange, deletion and/or addition from the sequence as shown in any one of SEQ ID NOs: 72, 73, 74, 75, 76, 77, 78, 79 or 80.

Preferably, the antibody according to the present invention comprises a light chain FR4 having an amino acid sequence selected from the group consisting of:
(a) an amino acid sequence as shown in any one of SEQ ID NOs: 103, 104, 105 or 106; and
(b) an amino acid sequence which differs by at least one amino acid exchange, deletion and/or addition from the sequence as shown in any one of SEQ ID NOs: 103, 104, 105 or 106.

It will be understood that a variant amino acid sequence which differs by at least one amino acid exchange, deletion and/or addition from any of the aforementioned amino acid sequences shall still be capable of exhibiting essentially the same immunological properties as the concrete amino acid sequence identified by a SEQ ID No. More preferably, such a variant amino acid sequence is at least 70%, at least 75%, at least 80%, at least 85%, at least 90%, at least 95%, at least 96%, at least 97%, at least 98%, at least 99% identical to the concrete amino acid sequence identified by a SEQ ID number.

More preferably, the antibody of the invention comprises in its heavy chain a combination of CDRs and FRs selected from the group consisting of:
SEQ ID NOs: 32, 53, and 81 for the CDRs and SEQ ID NOs: 22, 42, 63, and 99 for the FRs;
SEQ ID NOs: 33, 53, and 82 for the CDRs and SEQ ID NOs: 23, 43, 64, and 100 for the FRs;
SEQ ID NOs: 34, 54, and 83 for the CDRs and SEQ ID NOs: 24, 44, 65, and 100 for the FRs;
SEQ ID NOs: 35, 54, and 84 for the CDRs and SEQ ID NOs: 25, 44, 66, and 101 for the FRs;
SEQ ID NOs: 33, 56, and 85 for the CDRs and SEQ ID NOs: 26, 45, 67, and 100 for the FRs;
SEQ ID NOs: 36, 53, and 86 for the CDRs and SEQ ID NOs: 22, 42, 63 and 99 for the FRs;
SEQ ID NOs: 33, 57, and 87 for the CDRs and SEQ ID NOs: 27, 46, 68, 102 for the FRs;
SEQ ID NOs: 33, 56, and 85 for the CDRs and SEQ ID NOs: 26, 45, 67, and 100 for the FRs;
SEQ ID NOs: 32, 58, and 89; for the CDRs and SEQ ID NOs: 22, 47, 70, and 100 for the FRs; and
SEQ ID NOs: 37, 59 and 90 for the CDRs and SEQ ID NOs: 28, 48, 71, and 100 for the FRs.

More preferably, the antibody of the invention comprises in its light chain a combination of CDRs and FRs selected from the group consisting of:
SEQ ID NOs: 38, 60, and 91 for the CDRs and SEQ ID NOs: 29, 49, 72, and 103 for the FRs;
SEQ ID NOs: 38, 60, and 92 for the CDRs and SEQ ID NOs: 29, 49, 73, and 104 for the FRs;
SEQ ID NOs: 39, 61, and 93 for the CDRs and SEQ ID NOs: 30, 50, 74, and 105 for the FRs;
SEQ ID NOs: 40, 60, and 94 for the CDRs and SEQ ID NOs: 29, 49, 75, and 106 for the FRs;
SEQ ID NOs: 38, 60, and 95 for the CDRs and SEQ ID NOs: 29, 51, 76, and 106 for the FRs;
SEQ ID NOs: 38, 60, and 96 for the CDRs and SEQ ID NOs: 29, 49, 77, and 103 for the FRs;
SEQ ID NOs: 41, 62, and 97 for the CDRs and SEQ ID NOs: 52, 78, and 106 for the FRs;
SEQ ID NOs: 38, 60, and 95 for the CDRs and SEQ ID NOs: 29, 49, 79, and 106 for the FRs;
SEQ ID NOs: 38, 60, and 96 for the CDRs and SEQ ID NOs: 31, 49, 72, and 103 for the FRs;
SEQ ID NOs: 38, 60, and 94 for the CDRs and SEQ ID NOs: 29, 49, 72, and 106 for the FRs; and
SEQ ID NOs: 38, 60 and 97 for the CDRs and SEQ ID NOs: 29, 49, 80, and 106 for the FRs.

An antibody according to the invention may also be a full-length antibody (i.e. antibody comprising two heavy chains and two light chains). In such a case, the light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions. Occasionally, residues from non-hypervariable or framework regions (FR) influence the overall domain structure and hence the combining site. The light chains of human antibodies generally are classified as kappa and lambda light chains, and each of these contains one variable region and one constant domain. Heavy chains are typically classified as mu, delta, gamma, alpha, or epsilon chains, and these define the antibody's isotype as IgM, IgD, IgG, IgA, and IgE, respectively. Human IgG has several subtypes, including, but not limited to, lgG1, lgG2, lgG3, and lgG4. Human IgM subtypes include IgM, and lgM2. Human IgA subtypes include lgA1 and lgA2. In humans, the IgA and IgD isotypes contain four heavy chains and four light chains; the IgG and IgE isotypes contain two heavy chains and two light chains; and the IgM isotype contains ten or twelve heavy chains and ten or twelve light chains. Antibodies according to the invention may be IgG, IgE, IgD, IgA, or IgM immunoglobulins or fragments thereof.

A humanized antibody according to the invention refers to immunoglobulin chains or fragments thereof (such as Fab, Fab′, F(ab′)2, Fv, or other antigen binding sub-sequences of antibodies), which contain minimal sequence (but typically, still at least a portion) derived from non-human immunoglobulin. For the most part, humanized antibodies are human immunoglobulins (the recipient antibody) in which CDR residues of the recipient antibody are replaced by CDR residues from a nonhuman species immunoglobulin (the donor antibody) such as a mouse, rat or rabbit having the desired specificity, affinity and capacity. As such, at least a portion of the framework sequence of said antibody or fragment thereof may be a human consensus framework sequence. In some instances, Fv framework residues of the human immunoglobulin need to be replaced by the corresponding non-human residues to increase specificity or affinity. Furthermore, humanized antibodies can comprise residues which are found neither in the recipient antibody nor in the imported CDR or framework sequences. These modifications are made to further refine and maximize antibody performance. In general, the humanized antibody will comprise substantially all of at least one, and typically at least two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region, typically that of a human immunoglobulin, which (e.g. human) immunoglobulin constant region may be modified (e.g. by mutations or glycol-engineering) to optimize one or more properties of such region and/or to improve the function of the (e.g. therapeutic) antibody, such as to increase or reduce Fc effector functions or to increase serum half-life.

A chimeric antibody according to the invention refers to an antibody whose light and/or heavy chain genes have been constructed, typically by genetic engineering, from immunoglobulin variable and constant regions which are identical to, or homologous to, corresponding sequences of different species, such as mouse and human. Alternatively, variable region genes derive from a particular antibody class or subclass while the remainder of the chain derives from another antibody class or subclass of the same or a different species. It covers also fragments of such antibodies. For example, a typical therapeutic chimeric antibody is a hybrid protein composed of the variable or antigen-binding domain from a mouse antibody and the constant or effector domain from a human antibody, although other mammalian species may be used for both the constant and variable domains.

Preferably, the antibody according to the invention comprises a heavy chain having an amino acid sequence selected from the group consisting of:
(a) an amino acid sequence as shown in any one of SEQ ID NOs: 1, 2, 3, 4, 5, 6, 7, 9 or 10; and
(b) an amino acid sequence which differs by at least one amino acid exchange, deletion and/or addition from the sequence as shown in any one of SEQ ID NOs: 1, 2, 3, 4, 5, 6, 7, 9 or 10.

Preferably, the antibody according to the invention comprises a light chain having an amino acid sequence selected from the group consisting of:
(a) an amino acid sequence as shown in any one of SEQ ID NOs: 11, 12, 13, 14, 15, 16, 17, 19, 20 or 21; and
(b) an amino acid sequence which differs by at least one amino acid exchange, deletion and/or addition from the sequence as shown in any one of SEQ ID NOs: 11, 12, 13, 14, 15, 16, 17, 19, 20 or 21.

It will be understood that a variant amino acid sequence which differs by at least one amino acid exchange, deletion and/or addition from any of the aforementioned amino acid sequences shall still be capable of exhibiting essentially the same immunological properties as the concrete amino acid sequence identified by a SEQ ID No. More preferably, such a variant amino acid sequence is at least 70%, at least 75%, at least 80%, at least 85%, at least 90%, at least 95%, at least 96%, at least 97%, at least 98%, at least 99% identical to the concrete amino acid sequence identified by a SEQ ID number.

Particular preferred antibodies according to the present invention comprise a combination of a heavy chain and a light chain selected from the group of combinations consisting of:
SEQ ID NOs: 1 and 11 (FenAb136);
SEQ ID NOs: 2 and 12 (FenAb709);
SEQ ID NOs: 3 and 13 (FenAb156);
SEQ ID NOs: 4 and 14 (FenAb913);
SEQ ID NOs: 5 and 15 (FenAb196);
SEQ ID NOs: 6 and 16 (FenAb24);
SEQ ID NOs: 7 and 17 (FenAb440);
SEQ ID NOs: 5 and 19 (FenAb208)
SEQ ID NOs: 9 and 20 (FenAb609); and
SEQ ID NOs: 10 and 21 (FenAb861).

The term “hapten” as used herein refers to a small molecule, i.e. fentanyl or a derivative thereof. Such small molecules, typically, due to their size and other properties do not elicit an immune response in a physiological environment. However, it is possible by using the techniques according to the present invention to generate antibodies against said haptens that specifically bind such haptens. Moreover, upon binding the antibodies may also neutralize some or all biological effects caused by the small molecule haptens. Preferably, it is envisaged that the antibody of the invention specifically binds to fentanyl or a derivative thereof and thereby neutralizes said fentanyl or derivative thereof such that it can no longer exerts its pharmacological activities in an organism.

The term “fentanyl” as used herein refers to the compound N-phenyl-N-[1-(2-phenylethyl)piperidin-4-yl] propanamide (CAS number 437-38-7). Fentanyl is an opioid typically used as a pain therapeutic or for anesthesia. It is also abused as a recreation drug and may cause drug addiction. Fentanyl can be administered via different routes, e.g., by injection, nasal spray, transdermal (e.g., by skin patches), trans-mucosal, as a lozenge or tablet. Derivatives of fentanyl envisaged in accordance with the present invention comprise structurally and/or functionally related derivatives of fentanyl. Typically, fentanyl derivatives in accidence with the present invention are alfentanil, sufentanil, remifentanil, carfentanil, acetylfentanil and norfentanil. Preferably, the fentanyl derivative is carfentanil. Preferably, the hapten envisaged according to the invention is fentanyl.

The phrase “specifically binds to” as used in accordance with the present invention means that the antibody shall not cross-react significantly with components other than the hapten fentanyl or fentanyl derivatives. Cross-reactivity of an antibody as mentioned herein can be tested by the skilled person by various techniques including immunological technologies such as Western blotting, ELISA or RIA based Assays or measuring of binding affinities using, e.g., Biacore technology. Preferably, the antibody of the invention does not specifically bind to naloxone and/or tramadol.

The term “equilibrium dissociation constant (Kd)” as used herein indicates the propensity for the antibody/hapten complex to dissociate into its free components, i.e. free antibody and free hapten compound. The equilibrium dissociation constant (Kd) can be expressed as follows:

Kd=[A]ₓ*[B]_{y}/[AₓB_{y}]

wherein [Aₓ], [B_{y}], and [AₓB_{y}] are the concentrations of A, B and AB at the equilibrium, respectively. Thus, the smaller the equilibrium dissociation constant, the more tightly bound the ligand is, or the higher the affinity between ligand and protein. For example, a ligand with a pico-molar (pM) dissociation constant binds more tightly to a particular protein than a ligand with a nano-molar (nM) dissociation constant. The binding of the antibody of the invention and the hapten, i.e. fentanyl or a derivative thereof, shall be with an equilibrium dissociation constant (Kd) of at most 1.000 pM, at most 800 pM, at most 600 pM, at most 400 pM, at most 200 pM, at most 100 pM or at most 75 pM. The equilibrium dissociation constant referred to in accordance with the present invention can be determined by techniques well known in the art, preferably, it is to be determined using an ELISA described in the accompanying Examples, below.

Moreover, the antibody of the invention shall be capable of protecting mice, when being administered thereto, from adverse fentanyl actions when administered at a dosage (antibody compound (mg)/mouse body weight (kg)) of at most 10 mg/kg, at most 8 mg/kg, at most 6 mg/kg, at most 5 mg/kg, at most 4 mg/kg at most 3 mg/kg at most 2 mg/kg or at most 1 mg/kg.

Adverse fentanyl actions in mice as referred to in this context of the present invention are, preferably, an impairment of nociception. More preferably, impairment of nociception can be tested by applying a hot stimulus to mice that have been received fentanyl. Mice that are pre-treated by the antibody of the invention are protected from the adverse fentanyl actions and will exhibit restored or partially restored nociception while fentanyl treated mice that are not pre-treated by the antibody will exhibit impaired nociception caused by fentanyl. Impaired nociception is typically measured by determining the latency of a reaction of a mouse upon applying a hot temperature stimulus, e.g., using the hot plate test. Suitable test for measuring nociception are also well known in the art. Protection as referred to in this context of the present invention means that mice which received the antibody of the invention are either fully or partially protected from those adverse fentanyl actions, i.e. they shall react essentially as control mice that have not received fentanyl. The protection may be full protection or partial protection to a statistically significant extent. The dosage to be administered to mice can be provided via conventional routes of administration such as injection intra peritoneal. Preferably, the dosage is administered once prior to the administration of fentanyl. Typically, there is a time window of about 24 h between the administration of the antibody of the invention and the administration of fentanyl. Fentanyl is typically administered in a dosage of about 0.1 mg/kg. Preferably, the aforementioned protection of mice from adverse fentanyl actions can be tested as described in the accompanying Examples below or in Smith 2019.

Advantageously, it has been found in accordance with the studies underlying the present invention that anti-fentanyl antibodies can be generated which are capable of specifically binding to fentanyl with a particular high affinity with Kds in the pico-molar range. These antibodies allow for neutralizing fentanyl and, thus, for preventing or treating its adverse effects even if administered at rather low dosage. Using the technology for developing antibodies recognizing haptens such as fentanyl, the invention also enables the development of antibodies that specifically bind to fentanyl derivatives with particular high affinity, typically, with an affinity in the pico-molar range, and which require low dosage for eliciting protection.

Thanks to the present invention, therapeutic fentanyl use can be improved since its adverse side effects can be treated or prevented. Moreover, fentanyl abuse can be treated and/or prevented as well.

The present invention also relates to a polynucleotide encoding the antibody of the present invention.

The term “polynucleotide” as used in accordance with the present invention refers to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). The term as used herein encompasses the sequence specified herein as well as the complementary or reverse-complementary sequence thereof. Preferably, the polynucleotide is RNA or DNA. The term also encompasses DNAs or RNAs with backbones modified for stability or for other reasons. Moreover, DNAs or RNAs comprising unusual bases, such as inosine, or modified bases, such as tritylated bases, to name just two examples, are also encompassed as polynucleotides. It will be appreciated that a great variety of modifications have been made to DNA and RNA that serve many useful purposes known to those of skill in the art. Every nucleic acid sequence herein that encodes a certain polypeptide of the invention may due to the degeneracy of the genetic code have silent variations. The degeneracy of the genetic code yields a large number of functionally identical polynucleotides that encode the same polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are silent variations.

The polynucleotide of the invention shall encode the antibody of the invention, i.e. it shall comprise a nucleic acid sequences which encodes said antibody of the invention. In addition, the polynucleotide of the present invention may comprise additional nucleic acid sequences. Preferably, the polynucleotide of the present invention may comprise in addition to an open reading frame further untranslated sequence at the 3′ and at the 5′ terminus of the coding gene region: at least 500, preferably 200, more preferably 100 nucleotides of the sequence upstream of the 5′ terminus of the coding region and at least 100, preferably 50, more preferably 20 nucleotides of the sequence downstream of the 3′ terminus of the coding gene region.

The polynucleotide of the present invention shall be provided, preferably, either as an isolated polynucleotide (i.e. purified or at least isolated from its natural context such as its natural gene locus) or in genetically modified or exogenously (i.e. artificially) manipulated form. An isolated polynucleotide can, for example, comprise less than approximately 5 kb, 4 kb, 3 kb, 2 kb, 1 kb, 0.5 kb or 0.1 kb of nucleotide sequences which naturally flank the nucleic acid molecule in the genomic DNA of the cell from which the nucleic acid is derived. The polynucleotide, preferably, is provided in the form of double or single stranded molecule. It will be understood that the present invention by referring to any of the aforementioned polynucleotides of the invention also refers to complementary or reverse complementary strands of the specific sequences or variants there-of referred to before. The polynucleotide encompasses DNA, including cDNA and genomic DNA, or RNA polynucleotides.

Moreover, comprised are also chemically modified polynucleotides including naturally occurring modified polynucleotides such as glycosylated or methylated polynucleotides or artificial modified ones such as biotinylated polynucleotides.

The invention relates to a vector or expression construct comprising the polynucleotide of the present invention.

The term “vector”, preferably, encompasses phage, plasmid, cosmids, viral vectors as well as artificial chromosomes, such as bacterial or yeast artificial chromosomes (YAC). The vector encompassing the polynucleotide of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, it is to be understood that the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, f-mating, natural competence, carbon-based clusters, chemically mediated transfer, electroporation or particle bombardment. Suitable methods for the transformation or transfection of host cells, including plant cells, can be found in text books such as Sambrook et al. (Molecular Cloning: A Laboratory Manual, 2nd ed., Cold Spring Harbor Laboratory, Cold Spring Harbor Laboratory Press, Cold Spring Harbor, N Y, 1989). Alternatively, a plasmid vector may be introduced by heat shock or electroporation techniques. Should the vector be a virus, it may be packaged in vitro using an appropriate packaging cell line prior to application to host cells.

Preferably, the vector of the present invention is an expression vector. In such an expression vector, i.e. a vector which comprises the polynucleotide of the invention having the nucleic acid sequence operatively linked to an expression control sequence (also called “expression cassette”) allowing expression in prokaryotic or eukaryotic cells or isolated fractions thereof. Suitable expression vectors are known in the art such as Okayama-Berg cDNA expression vector pcDV1 (Pharmacia), pCDM8, pRc/CMV, pcDNA1, pcDNA3 (Invitrogene) or pSPORT1 (GIBCO BRL). Further examples of typical fusion expression vectors are pGEX, pMAL (New England Biolabs, Beverly, MA) and pRIT5 (Pharmacia, Piscataway, NJ), where glutathione S transferase (GST), maltose E-binding protein and protein A, respectively, are fused with the recombinant target protein. Examples of suitable inducible non-fusion E. coli expression vectors are, inter alia, pTrc and pET 11d. The target gene expression of the pTrc vector is based on the transcription from a hybrid trp-lac fusion promoter by host RNA polymerase. The target gene expression from the pET 11d vector is based on the transcription of a T7-gn10-lac fusion promoter, which is mediated by a co-expressed viral RNA polymerase (T7 gn1). This viral polymerase is provided by the host strains BL21 (DE3) or HMS174 (DE3) from a resident lambda-prophage which harbors a T7 gn1 gene under the transcriptional control of the lacUV 5 promoter. The skilled worker is familiar with other vectors which are suitable in prokaryotic organisms; these vectors are, for example, in E. coli, pLG338, pACYC184, the pBR series such as pBR322, the pUC series such as pUC18 or pUC19, the M113mp series, pKC30, pRep4, pHS1, pHS2, pPLc236, pMBL24, pLG200, pUR290, pIN-III113-B1, lambdagt11 or pBdC1, in Streptomyces p1J101, p1J364, p1J702 or p1J361, in Bacillus pUB110, pC194 or pBD214, in Corynebacterium pSA77 or pAJ667. Examples of vectors for expression in the yeast S. cerevisiae comprise pYep Sec1, pMFa, pJRY88 and pYES2 (Invitrogen Corporation, San Diego, CA). Vectors and processes for the construction of vectors which are suitable for use in other fungi, such as the filamentous fungi, comprise those which are described in detail in text books such as van den Hondel, C. A. M. J. J., & Punt, P. J. (1991) “Gene transfer systems and vector development for filamentous fungi, in: Applied Molecular Genetics of fungi, J. F. Peberdy et al., Ed., pp. 1-28, Cambridge University Press: Cambridge, or in: More Gene Manipulations in Fungi (J. W. Bennett & L. L. Lasure, Ed., pp. 396-428: Academic Press: San Diego). Further suitable yeast vectors are, for example, pAG-1, YEp6, YEp13 or pEMBLYe23. As an alternative, the polynucleotides of the present invention can be also expressed in insect cells using baculovirus expression vectors. Baculovirus vectors which are available for the expression of proteins in cultured insect cells, e.g., Sf9 cells, comprise the pAc series and the pVL series.

Yet the vector may be an integration vector. An integration vector refers to a DNA molecule, linear or circular, that can be incorporated, e.g., into a microorganism's genome, such as a bacteria's genome, and provides for stable inheritance of a gene encoding a polypeptide of interest, such as the alcohol acyl transferase of the invention. The integration vector generally comprises one or more segments comprising a gene sequence encoding a polypeptide of interest under the control of additional nucleic acid segments that provide for its transcription.

Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is non-functional in that organism. Integration of the segment comprising the gene of interest may be selected if an appropriate marker is included within that segment. One or more nucleic acid sequences encoding appropriate signal peptides that are not naturally associated with a polypeptide to be expressed in a host cell of the invention can be incorporated into (expression) vectors. For example, a DNA sequence for a signal peptide leader can be fused in-frame to a nucleic acid of the invention so that the alcohol acyl transferase of the invention is initially translated as a fusion protein comprising the signal peptide. Depending on the nature of the signal peptide, the expressed polypeptide will be targeted differently. A secretory signal peptide that is functional in the intended host cells, for instance, enhances extracellular secretion of the expressed polypeptide. Other signal peptides direct the expressed polypeptide to certain organelles, like the chloroplasts, mitochondria and peroxisomes. The signal peptide can be cleaved from the polypeptide upon transportation to the intended organelle or from the cell. It is possible to provide a fusion of an additional peptide sequence at the amino or carboxyl terminal end of the polypeptide.

The term “gene construct” as used herein refers to polynucleotides comprising the polynucleotide of the invention and additional functional nucleic acid sequences. A gene construct according to the present invention is, preferably, a linear DNA molecule. Typically, a gene construct in accordance with the present invention may be a targeting construct which allows for random or site-directed integration of the targeting construct into genomic DNA. Such target constructs, preferably, comprise DNA of sufficient length for either homologous or heterologous recombination as described in detail below. In both cases, the construct must be, preferably, impeccable, with structures to control gene expression, such as a promoter, a site of transcription initiation, a site of polyadenylation, and a site of transcription termination.

The present invention further relates to a host cell comprising the polynucleotide or the vector or expression construct of the present invention.

The host cell of the invention is capable of expressing the polypeptide of the invention comprised in the vector or gene construct of the invention. The host cell is, typically transformed with said vector or gene construct such that the polypeptide of the invention can be expressed from the vector or gene construct. The transformed vector or gene construct may be maintained as a non-integrated vector, for example, a plasmid, or alternatively, may be integrated into the host cell genome as specified elsewhere herein in more detail. A host cell according to the invention may be produced based on standard genetic and molecular biology techniques that are generally known in the art, e.g., as described in standard text books such as Sambrook, J., and Russell, D. W. “Molecular Cloning: A Laboratory Manual” 3d ed., Cold Spring Harbor Laboratory Press, Cold Spring Harbor, N Y, (2001); and F. M. Ausubel et al, eds., “Current protocols in molecular biology”, John Wiley and Sons, Inc., New York (1987), and later supplements thereto.

Preferably, said host cell is a bacterial cell, a fungal cell, an animal cell or a plant cell.

Bacterial cells may be gram-positive or gram-negative bacterial cells. Preferred bacterial cells may be selected from the genera Escherichia, Klebsiella, Helicobacter, Bacillus, Lactobacillus, Streptococcus, Amycolatopsis, Rhodobacter, Pseudomonas, Paracoccus, Lactococcus or Pantoea. More preferably, useful gram positive bacterial host cells may be Bacillus alkalophius, Bacillus amyloliquefaciens, Bacillus brevis, Bacillus circulans, Bacillus clausii, Bacillus coagulans, Bacillus firmus, Bacillus Jautus, Bacillus lentus, Bacillus licheniformis, Bacillus megaterium, Bacillus pumilus, Bacillus stearothermophilus, Bacillus subtilis, Bacillus thuringiensis, Streptomyces spheroides, Streptomyces thermoviolaceus, Streptomyces lividans, Streptomyces murinus, Streptoverticillum verticillium ssp. verticillium. Rhodobacter sphaeroides, Rhodomonas palustri, or Streptococcus lactis. Also more preferably, useful gram negative bacterial host cells may be Escherichia coli, Pseudomonas sp., preferably, Pseudomonas purrocinia, Pseudomonas fluorescens, Rhodobacter capsulatus, Rhodobacter sphaeroides, Paracoccus carotinifaciens, Paracoccus zeaxanthinifaciens or Pantoea ananatis.

Preferred fungal host cells may be Aspergillus, Fusarium, Trichoderma, Yeast, Pichia, or Saccharomyces host cells. Yeast as used herein includes ascosporogenous yeast, basidiosporogenous yeast, and yeast belonging to the Blastomycetes.

Preferred animal host cells may comprise mammalian host cells, avian host cells, reptilian host cells or insect host cells. Preferred animal host cells are HeLa cells, HEK293T cells, U2OS cells, A549 cells, HT1080 cells, CAD cells, P19 cells, NIH3T3 cells, L929 cells, N2a cells, CHO cells, MCF-7 cells, Y79 cells, SO-Rb50 cells, HepG2 cells, DUKX-X11 cells, J558L cells or BHK cells.

Preferred plant host cells comprise tobacco, rice, wheat, pea or tomato cells.

The present invention relates to a non-human transgenic organism comprising the polynucleotide or the vector or expression construct of the invention.

The term “non-human transgenic organism” as used herein refers to an organism which has been genetically modified in order to comprise the polynucleotide, vector or gene construct of the present invention. Said genetic modification may be the result of any kind of homologous or heterologous recombination event, mutagenesis or gene editing process. Accordingly, the transgenic nonhuman organism shall differ from its non-transgenic counterpart in that it comprises the non-naturally occurring (i.e. heterologous) polynucleotide, vector or gene construct in its genome. Non-human organisms envisaged as transgenic non-human organisms in accordance with the present invention are, preferably, multi-cellular organisms, such as an animal, plant, multi-cellular fungi or algae. Preferably, said non-human organism is an animal or a plant. Preferred animals are mammals, in particular, laboratory animals such as rodents, e.g., mice, rats, rabbits or the like, or farming animals such as sheep, goat, cows, horses or the like. Preferred plants are crop plants or vegetables, in particular, selected from the group consisting of tobacco, rice, wheat, pea and tomato. Methods for the production of transgenic non-human organisms are well known in the art; see, standard text books, e.g. Lee-Yoon Low et al., Transgenic Plants: Gene constructs, vector and transformation method. 2018. D01.10.5772/intechopen.79369; Pinkert, C. A. (ed.) 1994. Transgenic animal technology: A laboratory handbook. Academic Press, Inc., San Diedo, Calif.; Monastersky G. M. and Robl, J. M. (ed.) (1995) Strategies in Transgenic Animal Science. ASM Press. Washington D. C); Sambrook, loc.cit, Ausubel, loc.cit).

The present invention, in general, contemplates an antibody as defined before or a polynucleotide as defined before for use in treating and/or preventing a disease or condition in a subject associated with administration fentanyl or a derivative thereof. Preferably, the fentanyl derivative is selected from the group consisting of carfentanil, acetylfentanil, alfentanil, sufentanil, remifentanil, or norfentanil. More preferably, the fentanyl derivative is carfentanil.

The antibody or polynucleotide according to the present invention may be formulated as a medicament for use in in treating and/or preventing a disease or condition in a subject associated with administration fentanyl or a derivative thereof. Such a medicament is, preferably, for topical or systemic administration. Conventionally a medicament will be administered intra-muscularly or subcutaneously. However, depending on the nature and the desired therapeutic effect and the mode of action, the medicament may be administered by other routes as well. The medicament is, preferably, administered in conventional dosage forms prepared by combining the ingredients with standard pharmaceutical carriers according to conventional procedures. These procedures may involve mixing or dissolving the ingredients as appropriate to the desired preparation. Preferably, a solution is envisaged for the medicament. It will be appreciated that the form and character of the pharmaceutical acceptable carrier is dictated by the amount of active ingredient with which it is to be combined, the route of administration and other well-known variables. A carrier must be acceptable in the sense of being compatible with the other ingredients of the formulation and being not deleterious to the recipient thereof. The pharmaceutical carrier employed may include a solid, a gel, or a liquid. Examples for solid carriers are lactose, terra alba, sucrose, talc, gelatin, agar, pectin, acacia, magnesium stearate, stearic acid and the like. Exemplary of liquid carriers are phosphate buffered saline solution, syrup, oil, water, emulsions, various types of wetting agents, are distilled water, physiological saline, Ringer's solutions, dextrose solution, and Hank's solution, and the like. Similarly, the carrier may include time delay material well known to the art, such as glyceryl mono-stearate or glyceryl distearate alone or with a wax. For polynucleotides, liposomal carriers or genetically engineered viruses may be considered as well. In particular, if a long term application of the antibody is envisaged, a genetically engineered virus may be administered that produces the antibody of the invention over a long period within an organism to be treated. Said suitable carriers comprise those mentioned above and others well known in the art, see, e.g., Remington's Pharmaceutical Sciences, Mack Publishing Company, Easton, Pennsylvania. In addition, the medicament may also include other carriers, adjuvants, or non-toxic, non-therapeutic, nonimmunogenic stabilizers and the like. It is to be understood that the formulation of a medicament takes place under GMP standardized conditions or the like in order to ensure quality, pharmaceutical security, and effectiveness of the medicament.

A therapeutically effective dosage of the antibody or polynucleotide of the invention refers to an amount to be used in medicament. A therapeutically effective dosage is an amount of the antibody or polynucleotide that prevents, ameliorates or treats the symptoms accompanying a disease or condition referred to in this specification. Therapeutic efficacy and toxicity of the compound can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. The dosage regimen will be determined by the attending physician and other clinical factors. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. The medicament referred to herein is administered at least once in order to treat or ameliorate or prevent a disease or condition recited in this specification. However, the said medicament may be administered more than one time.

The term “treating” as used herein refers to any improvement, cure or amelioration of the disease or condition as referred to herein. It will be understood that treatment may not occur in 100% of the subjects to which the antibody has been administered. The term, however, requires that the treatment occurs in a statistically significant portion of subjects (e.g. a cohort in a cohort study). Whether a portion is statistically significant can be determined without further ado by a person skilled in the art using various well-known statistic evaluation tools, e.g., determination of confidence intervals, p-value determination, Student's t-test, Mann-Whitney-U test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.05, 0.01, 0.005, 0.001, or 0.0001.

The term “preventing” as used herein refers to significantly reducing the likelihood with which the disease or condition develops in a subject within a defined window (prevention window) starting from the administration of the antibody onwards. Typically, the prevention window is within 1 to 5 days, within 1 to 3 weeks, within 1 to 3 months. The prevention window depends on the amount of antibody or polynucleotide which is administered and the applied dosage regimen. Typically, suitable prevention windows can be determined by the clinician based on the amount of antibody or polynucleotide to be administered and the dosage regimen to be applied without further ado. It will be understood that prevention may not occur in 100% of the subjects to which the antibody has been administered. The term, however, requires that the prevention occurs in a statistically significant portion of subjects (e.g. a cohort in a cohort study). Whether a portion is statistically significant can be determined without further ado by a person skilled in the art using various well-known statistic evaluation tools, e.g., determination of confidence intervals, p-value determination, Student's t-test, Mann-Whitney-U test etc. Details are described elsewhere herein.

The phrase “disease or condition in a subject associated with administration fentanyl or a derivative thereof” refers to drug addiction caused by abusive intake of fentanyl or a derivative thereof, intoxication due to an inadvertent intake of fentanyl or a derivative thereof or any side-effects caused by a medically intended intake of fentanyl or a derivative thereof. Various adverse side-effects are known being associated with the administration of fentanyl or a fentanyl derivative. Typically, fentanyl may cause abdominal pain, headache, fatigue, anorexia and weight loss, dizziness, nervousness, anxiety, depression, flu-like symptoms, dyspepsia (indigestion), shortness of breath, respiratory depression, bradycardia, vasodilation, wooden chest syndrome, muscle rigidity, hypoventilation, apnoea, and/or urinary retention. Intake of an overdose of fentanyl or a derivative thereof may also cause death.

The antibody of the invention can be, preferably, manufactured by the following method comprising the steps of:
a) contacting a splenic sample of an animal, preferably, a mouse, which has been immunized with the hapten fentanyl or a derivative thereof with labeled hapten;
b) isolating individual cells from that sample that:
   are CD19 positive;
   are CD138 negative;
   having bound the labeled hapten;
c) determining the nucleic acid sequences of a plurality of expressed genes, preferably, the entire transcriptome, for each of said isolated individual cells;
d) selecting individual memory B-cells among the individual isolated cells by identifying the presence of nucleic acid sequences of one or more expressed genes selected from the group consisting of: BhIhe41, Parm1, CD80, CobI, IgG1, IgG2A, IgG2B, IgG3, IgG4, IgA, IgE, Sspn, Ackr2, Nt5e, and Mki67 within the nucleic acid sequences of a plurality of expressed genes;
e) assembling antibody light and heavy variable chain encoding nucleic acid sequences from the nucleic acid sequence of the plurality of expressed genes of the selected individual memory B-cells; and
f) expressing the antibody light and heavy chain encoding nucleic acids assembled in step e) in a host cell in order to manufacture the antibody.

The term “manufacture” as used herein refers to the process of generation of the antibody which specifically recognizes the hapten starting the splenic sample of an animal which has been immunized by the hapten to the recombinant production of the antibody in a host cell. The manufacture may also comprise further steps such as purifying the produced antibody or formulating the antibody or purified antibody as a pharmaceutical composition. Accordingly, the aforementioned method of the present invention may consist of the aforementioned steps or may comprise further additional steps.

The phrase “specifically binds to” as used in accordance with the present invention means that the antibody shall not cross-react significantly with components other than the hapten. Cross-reactivity of an antibody as mentioned herein can be tested by the skilled person by various techniques including immunological technologies such as Western blotting, ELISA or RIA based Assays or measuring of binding affinities using, e.g., Biacore technology.

The term “labeled hapten” as used herein refers to hapten which is linked to a label that can be used for isolating the cell. Typically, a label as referred to herein is a fluorescent dye which can be determined by FACS, a magnetic label which can be determined by MACS or a label which can be determined in any other method for isolating single cells described herein. Preferably, a fluorescent dye which may be used in accordance with the present invention as a label for the hapten is (i) a single dye, such as DyLight 405, Alexa Fluor 405, Pacific Blue, Alexa Fluor 488, FITC, DyLight 550, PE, APC, Alexa Fluor 647, DyLight 650, PerCP, or Alexa Fluor 700, (ii) a starbright dye, such as StarBright Violet 440, 515, 610, or 670 or StarBright Blue 700, (iii) a tandem dye capable of FRET, such as PE-Alexa Fluor® 647, PE-Cy5, PerCP-Cy5.5, PE-Cy5.5, PE-Alexa Fluor® 750, PE-Cy7, or APC-Cy7, or (iv) a fluorescent protein such as EGFP, CFP, EGFP, YFP, RFP or mCHERRY. Preferably, a magnetic label which may be used in accordance with the present invention as a label for the hapten is a dynabead. The label may be linked to the hapten via a permanent or reversible linkage, i.e. it may be linked via a chemical bond or via reversible chemical interactions such as electrostatic interactions and the like. The label may be linked to the hapten by a linker molecule. Depending on the nature of the label and/or the hapten, the skilled person is well aware of which linkers may be used.

The term “contacting” as used herein refers to brining into physical proximity the labeled hapten and the cells comprised in the splenic sample such that cells which are able of specifically binding to the labeled hapten are capable of doing so. Accordingly, contacting is to be carried out for a time and under conditions which allow for specific binding of the labeled hapten to the said cells. Typically, the splenic sample is contacted for a time within the range of about 15 to about 60 min, preferably, about 30 min to about 45 min, more preferably, about 45 min. Typically, conditions for contacting are: (i) staining with a live/dead stain (e.g. Live/Dead Blue Dye from Thermo Scientific or Propidium iodide) to remove dead cells from the analysis (ii) blocking Fc receptors for 15 minutes on cells in order to prevent unspecific antibody binding; (iii) contacting with a decoy label (a conjugate of a fluorescent label and another fluorescent label of a different color, wherein the former is the same label that will be used in antigen-contacting in the following step) for 10 minutes, (vi) contacting with labeled hapten, e.g., fluorescent fentanyl at a 1:2000 dilution relative to the staining volume, for about 45 minutes; (v) staining with all primary B cell identification antibodies (e.g., anti-CD138-, anti-CD19-antibodies) for 45 minutes; (vi) staining with required secondary antibodies for 15 minutes. All steps are executed, preferably, on ice. Washing steps between the aforementioned steps (i) or (vi) may be performed as well including centrifugation and resuspension of the cellular pellet in a suitable washing solution. Most preferably, contacting is carried out as described in the accompanying Examples, below.

The term “splenic sample” as used herein refers to a sample derived from the spleen comprising antibody producing cells, preferably, different types of B-cells. The sample is, typically, a tissue sample which or may not be pre-treated in order to remove single cells from the splenic tissue. Preferably, the splenic sample is a homogenized total spleen sample. The skilled person is well aware of how such splenic samples can be obtained, e.g., by biopsy of parts of the spleen or by splenectomy.

The term “animal” as used herein refers to a non-human animal which is suitable for immunization and antibody production and from which splenic samples may be taken in order to isolate antibody-producing cells, preferably, different types of B-cells. Accordingly, the animal shall have a humoral immune system. Preferably, suitable animals are mammals, more preferably, laboratory animals such as rodents, most preferably, mice, or farming animals such as goat, sheep, pig or cow.

The term “isolating” as used herein refers to physically separating individual cells on a single cell level from the sample. Said isolating cells on a single cell level can be achieved by cell sorting techniques including, e.g., fluorescent activated cell sorting (FACS) or magnetic activated cell sorting (MACS). Typically, cells which are comprised in a sample are separated individually by cell sorting techniques based on the determination of labels which are present on the surface or within said cells. Other techniques may be based on microfluidic devices using different microfluidic channels into which cells can enter, e.g., by altering the flow path, or buoyancy activated cell sorting (BACS). Upon cell sorting has been carried out, the individual cells are, typically, maintained in a micro-well plate for further analysis.

The term “individual cells” as used herein refers to a collection of isolated, i.e. physically separated, single cells.

The term “CD19” as used herein refers to Cluster of Differentiation 19, a B-cell surface antigen which is a transmembrane protein expressed in all B lineage cells, including Plasma cells although in these cells it is downregulated. CD19 plays two major roles in B cells: (i) It acts as an adaptor protein to recruit cytoplasmic signaling proteins to the membrane, and (ii) It works within the CD19/CD21 complex to decrease the threshold for B-cell receptor signaling pathways. Due to its presence on all B cells, it is a biomarker for B-cell development, lymphoma diagnosis and can be utilized as a target for leukemia immunotherapies. The human CD19 protein is deposited under UniProt no.: P15391, mouse CD19 under UniProt no.: P25918. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before. Antibodies which specifically bind to CD19 are available in the prior art and are described, e.g., in Triller 2017, Immunity 47(6): 1197-1209 (human anti-CD19 antibody) or Cho 2018, Nat. Commun. 9(1): 2757 (mouse anti-CD19 antibody). They are commercially available from Thermo Fisher Scientific, US.

The term “CD138” or syndecan 1 as used herein refers to a transmembrane (type I) heparan sulfate proteoglycan and is a member of the syndecan proteoglycan family. The syndecan-1 protein functions as an integral membrane protein and participates in cell proliferation, cell migration and cellmatrix interactions via its receptor for extracellular matrix proteins. Syndecan-1 is a sponge for growth factors and chemokines, with binding largely via heparan sulfate chains. The syndecans mediate cell binding, cell signaling, and cytoskeletal organization and syndecan receptors are required for internalization of the HIV-1 tat protein. Altered syndecan-1 expression has been detected in several different tumor types. Syndecan 1 can be a marker for plasma cells. The human CD138 protein is deposited under UniProt no.: P18827, mouse CD19 under UniProt no.: P18828. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before. Antibodies which specifically bind to CD138 are available in the prior art and are described, e.g., in Cho 2018, Nat. Comun. 16(9): 2757. They are commercially available from Thermo Fisher Scientific, US.

The term “determining the nucleic acid sequences” as used herein refers to determining the order of nucleotides of the nucleic acids, i.e. their sequences. Said determining the nucleic acid sequence can be carried out by any known DNA or RNA sequencing technique including Sanger sequencing, pyrosequencing, next-generation sequencing, sequencing by reversible terminator chemistry, sequencing-by-ligation mediated by ligase enzymes, phosphor-linked fluorescent nucleotides or realtime sequencing, and the like. Various technology platforms are commercially available, e.g., from Roche, Illumina, or Life technologies. Preferably, sequencing of the nucleic acids is carried out by Illumina NGS and following the SMART seq 2.5 library preparation protocol developed by Picelli 2014, Nature Protocols 9, 171-181, and modified by the Single-cell Open Lab (scOpenLab).

The term “plurality” as used herein, generally, refers to a larger number of items such as the expressed genes referred to in accordance with the invention. A plurality in accordance with the present invention, thus, refers to at least 100, at least 1,000, at least 10,000, at least 100,000 or at least 1,000,000 expressed genes. More specifically, it is envisaged that the plurality of expressed genes corresponds to the entire detectable transcriptome, i.e. the entirety of expressed genes of a cell investigated by the method of the present invention that can be detected by sequencing.

The term “expressed genes” as used herein refers to any gene of a cell which is expressed by said cell, i.e. for which RNA, typically, mRNA, can be found in the cell. Contrary to the expressed genes, there are genes which are silent, i.e. which are only present in the genome of the cell but which are not expressed and for which, consequently, no RNA is to found in the cell.

The term “selecting” as used herein refers to identifying an isolated individual cell and the dataset obtained therefrom, e.g., the dataset comprising the nucleic acid sequences determined in said cell, and further evaluating said dataset of said cell by identifying the presence of nucleic acid sequences of one or more expressed genes selected from the group consisting of: BhIhe41, Parm1, CD80, CobI, IgG1, Sspn, Ackr2, Nt5e, and Mki67 within the nucleic acid sequences of a plurality of expressed genes.

The term “memory B-cells” as used herein refers to a dormant type of B-cell which is obtained as a result of cellular differentiation from naïve B-cells. Memory B-cells can differentiate into Plasma cells upon a second contact with an antigen. Said differentiation is typically faster than the differentiation of naïve B-cells into Plasma cells allowing for a faster humoral immune response in second time infections. Memory B-cells can survive for decades in in the organism and, thus, serve as a memory reservoir. Since B-cells have, typically, undergone class switching, they can express a range of immunoglobulin molecules. Memory B-cells that express IgM can be, typically, found concentrated in the tonsils, Peyer's patch, and lymph nodes. This subset of memory B-cells is more likely to proliferate and reenter the germinal center during a secondary immune response. Memory B-cells that express IgG typically differentiate into plasma cells. Memory B-cells that express IgE are very rare in healthy individuals. This may occur because B-cells that express IgE more frequently differentiate into plasma cells rather than memory B-cells. Memory B-cells that express IgD are very rare. B-cells with only IgD are found concentrated in the tonsils. Memory B-cells as referred to in accordance with the present invention shall typically exhibit the characteristic used for isolation from the splenic sample, i.e., they shall be CD19 positive, shall be CD138 negative, and shall be capable of specifically binding the labeled hapten. Moreover, the memory B-cells envisaged in accordance with the present invention shall express at least one biomarker selected from the group consisting of: BhIhe41, Parm1, CD80, CobI, IgG1, IgG2A, IgG2B, IgG3, IgG4, IgA, IgE, Sspn, Ackr2, Nt5e, and Mki67. More preferably, the memory B-cells envisaged in accordance with the present invention shall express all of the aforementioned biomarkers.

The term “BhIhe41” as used herein refers to a basic helix-loop-helix transcription factor repressor protein in various tissues of both humans and mice. BHLHE41 is known for its role in the circadian molecular mechanisms that influence sleep quantity as well as its role in immune function and the maturation of T helper type 2 cell lineages associated with humoral immunity. The human protein is deposited under UniProt no.: Q9C0J9, mouse protein under UniProt no.: Q99PV5. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “Parm1” as used herein refers to the Prostate androgen-regulated mucin-like protein 1. The human protein is deposited under UniProt no.: Q6UWI2, mouse protein under UniProt no.: Q923D3. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “CD80” as used herein refers to the Cluster of differentiation 80 (also CD80 and B7-1) is a B7, type I membrane protein in the immunoglobulin superfamily having an extracellular immunoglobulin constant-like domain and a variable-like domain required for receptor binding. CD80 can be found on the surface of various immune cells, including B-cells, monocytes, or Tcells, most typically at antigen-presenting cells (APCs), such as dendritic cells. CD80 has a crucial role in modulating T-cell immune function as a checkpoint protein at the immunological synapse. Expression of CD80 in B cells is associated with T cell dependent activation in the case of T dependent antigens. The human protein is deposited under UniProt no.: P33681, mouse protein under UniProt no.: Q00609. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “CobI” as used herein refers to the Cordon-bleu protein which was demonstrated to be a brain-enriched, Wiskott-Aldrich Homology 2 WH2 domain-based actin nucleator playing a pivotal role in morphogenetic processes in the vertebrate central nervous system (CNS) that give rise to the complex dendritic arbor of neuronal cells. The human protein is deposited under UniProt no.: 075128, mouse protein under UniProt no.: Q5NBX1. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The terms “IgG1”, “IgG2A”, “IgG2B”, “IgG3”, “IgG4”, “IgA”, and “IgE” as used herein refer to the corresponding immunoglobulins, e.g., IgG1 refers to immunoglobulin G1, etc. All these immunoglobulin subtypes are well known. Amino acid sequences for human and mouse immunoglobulin subtypes are well known and vary depending on the antigen target. Further details on Immunoglobulins or antibodies are also to be found elsewhere herein.

The term “Sspn” as used herein refers to sacrospan a K-ras associated polypeptide. It is a member of the dystrophin-glycoprotein complex which spans the sarcolemma and is comprised of dystrophin, syntrophin, alpha- and beta-dystroglycans and sarcoglycans. The human protein is deposited under Genbank accession number XP 011519155.1. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “Ackr2” as used herein refers to atypical chemokine receptor 2, a beta chemokine receptor, which is to be a seven transmembrane protein similar to G protein-coupled receptors. It is expressed in a range of tissues and hemopoietic cells. The expression of this receptor in lymphatic endothelial cells and overexpression in vascular tumors suggested its function in chemokine-driven recirculation of leukocytes and possible chemokine effects on the development and growth of vascular tumors. This receptor appears to bind the majority of beta-chemokine family members; however, its specific function remains unknown. The human protein is deposited under UniProt no.: 000590. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “Nt5e” as used herein refers to 5′-nucleotidase (5′-NT), also known as ecto-5′-nucleotidase or CD73 (cluster of differentiation 73). Nt5e is an enzyme is capable of converting AMP to adenosine. The human protein is deposited under UniProt no.: P21589, mouse protein under UniProt no.: Q61503. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “Mki67” as used herein refers to a nuclear protein which is associated with proliferation. The human protein is deposited under UniProt no.: P46013, mouse protein under UniProt no.: E9PVX6. It will be understood, however, that the term also encompasses variant proteins which differ from the proteins having the aforementioned amino acid sequences by at least one amino acid exchange, deletion and/or addition. Typically such variants, e.g., homologs, orthologs or paralogs, have an amino acid sequence which is at least 80%, at least 85%, at least 90%, at least 95%, at least 98% or at least 99% identical to the concrete sequences referred to before.

The term “assembling” as used herein refers to establishing the amino acid sequence of an antibody light and heavy chain from the determined nucleic acid sequences, i.e. the sequence dataset, of an individual isolated cell. Preferably, assembling refers to establishing at least the variable amino acid sequence of the light and heavy chains, more preferably, the entire light and heavy chain amino acid sequences based on the sequence dataset of an individual cell. The process of assembling said amino acid sequence of an antibody light and heavy chain may include using bioinformatics tools such as the BASIC software (Canzar 2019) described elsewhere herein and pre-compiled sequence data for variable and constant regions for facilitating and/or improving the assembling process.

The term “antibody light and heavy variable chain” as used herein refers to the immunoglobulin heavy chain (IgH) which is the large polypeptide subunit of an antibody. In the human genome, the IgH gene loci are on chromosome 14. An antibody is, typically, composed of two immunoglobulin (Ig) heavy chains and two Ig light chains that are the small polypeptide subunits of an antibody. Several different types of heavy chain exist that define the class or isotype of an antibody. The heavy chain types vary between different animals. All heavy chains contain a series of immunoglobulin domains, usually, with one variable domain (VH) that is important for binding antigen and several constant domains (CH1, CH2, etc.). Only one type of light chain is present in a typical antibody, thus the two light chains of an individual antibody are identical. Each light chain is composed of two tandem immunoglobulin domains, i.e., one constant (CL) domain and one variable domain (VL) which is important for binding the antigen. The antibody heavy and light chains assembled in the method of the present invention may be used as assembled or their amino acid sequences may be further modified in order to produce antibody derivatives such as humanized antibodies or chimeric antibodies.

The term “expressing” as used herein refers to transcribing and translating the nucleic acids encoding the antibody light and heavy chain in the host cell such that a functional antibody is produced and secreted from the host cell. A functional antibody as referred to in this context is an antibody which is capable of specifically recognizing the hapten.

In a preferred embodiment of the aforementioned method of the manufacture of the antibody of the present invention, said animal has been immunized with the hapten using an immunization method comprising the steps of:
(i) administering at least once an immunogenic particle exhibiting a plurality of hapten molecules as a priming step; and
(ii) administering at least once carrier molecules exhibiting single hapten molecules as a boosting step.

Preferably, it is envisaged to use antigenic particles exhibiting on its surface VSG proteins as carriers that may be coupled to the hapten by suitable chemical linkage. For the linking the hapten molecule to the VSG protein, coupling chemistry such as the “Click” chemistry may be used. Alternatively, modified VSG proteins may be used as carriers which allow for sortagging the hapten molecule to the VSG. Sortagging is an enzymatic coupling technique which is based on the linkage established between a sortagging donor peptide and a sortagging acceptor peptide by the enzyme sortase. The process is well established in the art and details are to be found, e.g., in WO2021/214043. Preferably, the immunogenic particle is selected from the group consisting of: a liposome, micelle, a bead, a vesicle and a cell. Preferably, the said cell is a trypanosome cell, preferably an inactivated, such as UV inactivated, trypanosome cell, or any membrane fragments thereof. The immunogenic particle shall carry via the carrier protein exhibited on its surface a plurality of hapten molecules, preferably, at least 5, at least 10, at least 20, at least 30, at least 40 or at least 50 hapten molecules. The immunogenic particle referred to herein is used for priming, i.e., the first administration of the hapten to be used for immunization. Priming may be carried out once or at several time points, typically, twice with 30 days in between both priming immunizations. In a boosting step, i.e. step b) of the aforementioned method for immunization, the hapten is administered without an immunogenic particle. Typically, the VSG carrier protein coupled to the hapten is administered in “soluble” form, i.e. not immobilized on a larger immunogenic particle. Boosting may be carried out once or at several time points, typically, twice with 30 days in between both boosting immunizations and 30 days between the last priming and the first boosting immunization. Further details on how to carry out the immunization may be found in WO2021/214043.

In a preferred embodiment of the aforementioned method of the manufacture of the antibody of the present invention, said isolating individual cells in step b) is carried out by using single cell sorting techniques. Preferably, FACS is used as a single cell sorting techniques, more preferably, FACS as described in the accompanying Examples, below.

In a preferred embodiment of the aforementioned method of the manufacture of the antibody of the present invention, wherein said isolating individual cells in step b) further comprises isolating individual cells that are viable cells. More preferably, a viable cell is negative for 7-aminoactinomycin (7AAD) staining. 7AAD is a double stranded DNA intercalating agent. It is known to enter and stain cells having defects in their cell membranes such as dead cells. Thus, 7AAD staining will result in staining of dead cells while the viable cells are not stained. Dead cells shall be, preferably, sorted out in the method of the invention. Sorting may, preferably, be carried out by FACS, more preferably, as described in the accompanying Examples, below.

In a preferred embodiment of the aforementioned method of the manufacture of the antibody of the present invention, said method further comprises determining whether the isolated cells in step b) exhibit IgG on its surface.

In a preferred embodiment of the aforementioned method of the manufacture of the antibody of the present invention, said determining the nucleic acid sequences of a plurality of expressed genes in step c) is carried out by using a single cell sequencing technique.

In a preferred embodiment of the aforementioned method of the manufacture of the antibody of the present invention, said determining the nucleic acid sequences of a plurality of expressed genes in step c) comprises a bioinformatic evaluation of the determined nucleic acid sequences. More preferably, said bioinformatic evaluation comprises generating datasets for each individual cell which contain data reflecting the in vivo expression profile.

Also more preferably, said generating datasets for each individual cell which contain data reflecting the expression profile comprises the steps of:
(i) removing low quality sequence reads;
(ii) removing adaptor sequences from carrying out single cell sequencing;
(iii) aligning nucleic acid sequences to an indexed reference genome;
(iv) compiling a BAM file for the plurality of nucleic acid sequences of expressed genes aligned to a reference genome for each individual cell, and allocating a quality score for the quality of the alignment of sequences to the indexed reference genome to each BAM file;
(v) removing low quality sequence reads based on the allocated quality score, preferably, Phred score;
(vi) annotating the aligned sequences of a BAM file to chromosomal loci;
(vii) compiling a count matrix dataset comprising identifier for the individual cells and identifier for the expressed genes;
(viii) removing low quality matrix datasets (representing individual cells) based on the following criteria: high percentage of mitochondrial genes, total number of nucleic acid sequence reads and number of expressed genes in an individual cell; and
(ix) removing individual genes that are significantly underrepresented;

The aforementioned steps may be, preferably, carried out as described in the following:

### (i) Removing Low Quality Sequence Reads

Generally, low quality sequence reads shall be removed at this step. Low quality sequences are those with nucleotide sequences that cannot be reliably/confidently called, or those that are too short for the eventual genome alignments to be reliable. This may be, preferably, performed by using bioinformatics tools such as the tools “trim galore (version 0.6.4 dev)” and “cutadapt (version 1.18)”.

(ii) Removing Adaptor Sequences from Carrying Out Single Cell Sequencing

The sequencing process (e.g., RNAseq) adds adapter sequences to all reads in order to allow the sequencing platform to recognize only the nucleic acid sequences that were generated by the sequencing PCRs. These adaptor sequences shall be removed bioinformatically, since they may otherwise disrupt the eventual genome alignments. This may also be, preferably, performed by using bioinformatics tools such as the tools “trim galore (version 0.6.4 dev)” and “cutadapt (version 1.18)”.

(iii) Aligning Nucleic Acid Sequences to an Indexed Reference Genome

As a result of the steps carried out before, a pool of high quality sequence read data for each cell is obtained. In a next step, the sequenced reads are to be aligned against a reference genome in order to identify the genes being expressed. To this end, each read is aligned against a reference genome (during said alignment, each read acquires the chromosomal coordinates of the reference genome they are aligned to). For this process, the bioinformatics tool “STAR” (Version STAR 2.6.0a) is typically used. For sequences originating from mouse samples, the “Release M25 (GRCm38.p6)” mouse genome, and for sequences originating from human samples, the “Release 38 (GRCh38.p13)” human genome can be, preferably, used as a reference genome. Genome indexing is a step conducted once, and the indexed genome can then be used for all future datasets for reads having the same sequencing length. However, indexing must be repeated if a new reference genome or version of the reference genome shall be used or if sequencing length of the reads changes.

(iv) Compiling a BAM File for the Plurality of Nucleic Acid Sequences of Expressed Genes Aligned to a Reference Genome for Each Individual Cell and Allocating a Quality Score for the Quality of the Alignment of Sequences to the Indexed Reference Genome to Each BAM File

The compilation of the plurality of nucleic acid sequences of expressed genes aligned to a reference genome for each individual cell shall generate genome-aligned “BAM” files for each cell that will be used in the next step. These files contain information for each individual read and its alignment to the genome, along with some quality measures, such as a Phred score.

The BAM files are then, typically, sorted and filtered to make the downstream steps easier to do. This process may be performed using the bioinformatics tool “Samtools” (Version Samtools 1.9). The aligned transcripts at this point are mixed in the BAM files. Since the information for the chromosome location is known, the files can be sorted in order to group all the transcripts based on their chromosomal positioning. This will facilitate the subsequent filtering step of the files.

### (v) Removing Low Quality Sequence Reads Based on the Allocated Quality Score

At this stage, more low-quality sequences are removed which are recognized at this stage for their inability to align well-enough to the genome. These sequences are, typically, identified based on the Phred score, a quality score that was calculated in step (iii), e.g., during the STAR alignment. Furthermore, at this stage, duplicated reads are removed. These reads have identical chromosomal coordinates and are probably the result of a PCR bias. Their presence does not necessarily reflects an actual enrichment on the mRNA levels and are for these reasons removed. Moreover, reads that are not mapped to any position are removed, too.

### (vi) Annotating the Aligned Sequences of a BAM File to Chromosomal Loci

Next, the aligned reads in the BAM files need to be annotated (the reads identified as mapping to chromosome 1, position X need to be mapped instead to an identified gene ID). This requires the use of a number of different tools, listed in the notes. The skilled person is well aware of several options to use. For example, the exons, the intron regions, or both may be mapped. Preferably, the exons may be used.

(vii) Compiling a Matrix Dataset Comprising Identifier for the Individual Cells and Identifier for the Expressed Genes

At this point, with the ID of the cell in each column and the ID of the genes in each row a matrix of the data has been created. The values of the matrix is the number of reads belonging to each gene for each cell.

(viii) Removing Low Quality Matrix Datasets (Representing Individual Cells) Based on the Following Criteria: High Percentage of Mitochondrial Genes, Total Number of Nucleic Acid Sequence Reads and Number of Expressed Genes in an Individual Cell

Next, individual cells (columns) of the count matrix that are of poor quality are eliminated. These cells are identified as “bad quality cells” for the following reasons: Cell with a high percentages (Z) of transcript reads mapped and annotated as mitochondrial genes. This is an indication that cell is undergoing cell death. It will not be informative for the further analysis and, in particular, characterizing B cell developmental stage (because the genes used to identify memory B cells are genomic, not mitochondrial). Keeping them might also gravitate the analysis towards the wrong direction and characterize falsely some populations. Additional quality control metrics, are the library size (total number of transcripts/reads) and the number of genes identified in each cell. Cells that display less than (X) total “features” (individual genes annotated in the transcriptome) and less than (Y) number of reads are filtered out under the predilection that they are “incomplete” and thus not informative enough to carry statistical relevance. X, Y and Z are calculated for each cell, and then outlier-cells (based on the median absolute deviation (MDA=3) for X, Y and Z) are removed.

(ix) Removing Data on Individual Genes that are Significantly Underrepresented

Next, individual annotated genes are eliminated from the remaining individual transcriptome datasets on the basis of representation. For example, a cell with a “good” overall transcriptome (assessed in the previous step) may still have certain genes that are not represented well enough to be considered statistically relevant. E.g., a gene that is annotated as present in an individual cell's transcriptome, but only has one or two reads (the actual cutoff being applied is 3 reads in less than 5 cells), that gene and its reads will be eliminated. Further, at this stage, transcripts that map to the immunoglobulin (antibody) genes are eliminated. This is because antibody transcripts are very abundant in B cell transcriptomes, and, thus, make the overall transcriptome unfairly weighted.

In a preferred embodiment of the aforementioned method of the manufacture of the antibody of the present invention, said bioinformatic evaluation comprises cluster analysis of the individual cells based on the datasets for each individual cell which contain data reflecting the expression profile. More preferably, said cluster analysis comprises the steps of:
(i) normalizing the expression levels for each gene between the datasets of the individual cells by deconvolution;
(ii) identifying the genes based on which the clustering will be made by modelling the variability of the gene expression of the expressed genes of a cell;
(iii) performing a dimension reduction analysis, preferably, a principal component analysis for the datasets of the individual cells resulting in clustering of the individual cells in different subpopulations based on features derived from said datasets;
(iv) integrating RNAseq datasets from different sequencing plates and clustering B-cells in different subpopulations with integrative non negative matrix factorization; and
(v) identifying individual memory B-cells as cells from a subpopulation of cells identified by clustering.

The aforementioned steps may be, preferably, carried out as described in the following:

### (i) Normalizing the Expression Levels for Each Gene Between the Datasets of the Individual Cells by Deconvolution

Now that overall transcriptomes have been generated, the cells need to be clustered to identify separable B cell populations. A first step towards this direction is to normalize the level of expression for each gene in cell. The goal is to correct for the cell-to-cell differences, remove cell specific biases and ensure that downstream cell to cell comparison of relative expression is valid. There are many possible ways to normalize the gene expression. Towards the identification of rare populations, all biological variability should be maintained post-normalization. Preferably, normalization is achieved by deconvolution. Typically, a pool of cells is selected and the expression profiles for those cells are summed together. “The pooled expression profile is normalized against an average reference pseudo-cell which is constructed by averaging the counts across all cells. This defines a size factor for the pool as the median ratio between the count sums and the average across all genes. The scaling bias for the pool is equal to the sum of the biases for the constituent cells. The same applies for the size factors, as these are effectively estimates of the bias for each cell. Repeating this process for multiple pools will yield a linear system that can be solved to obtain the size factors for the individual cells. In this manner, pool-based factors are deconvolved to yield the relevant cell-based factors. Any systematic difference in expression across the majority of genes is treated as bias, which is incorporated into the size/normalization factors and removed upon scaling. Size factors computed within each cluster must be rescaled for comparison between clusters. This is done by normalizing between the per-cluster pseudo-cells to identify the rescaling factor. One cluster is chosen as a “reference” to which all others are normalized. Ideally, the reference cluster should have a stable expression profile and not be extremely different from all other clusters. The assumption here is that there is a non-DE majority between the reference and each other cluster by Aaron Lun and Karsten Bach (Lun 2016, Genome Biol. 17:75). After this, a log transformation on the normalized expression values may be applied.

(ii) Identifying the Genes Based on which the Clustering Will be Made by Modelling the Variability of the Gene Expression of the Expressed Genes of a Cell;

At this step, each cell has a plurality of (approximately 20,000) genes in a satisfying normalized expression level. Many of them are housekeeping genes or are expressed at the same level for all the cells. These genes are not important to group together cells with similar profiles, are they are expressed by all the cells. A way to measure the level of variability for a gene is to calculate the variance and mean of the log expression. More specifically, “for each gene, the variance and mean of the log-expression values was calculated. A trend is fitted to the variance against the mean for all genes. The fitted value for each gene is used as a proxy for the technical component of variation for each gene under the assumption that most genes exhibit a low baseline level of variation that is not biologically interesting. The biological component of variation for each gene is defined as the residual from the trend. Ranking genes by the biological component enables identification of interesting genes for downstream analyses in a manner that accounts for the mean-variance relationship. Log-transformed expression values can be used to blunt the impact of large positive outliers and to ensure that large variances are driven by strong log-fold changes between cells rather than differences in counts. Log-expression values are also used in downstream analyses like PCA, so modelling them here avoids inconsistencies with different quantifications of variation across analysis steps. After this step, the cells that reached this step of the analysis and the genes above the mean of variance may be used to cluster the cells in different subpopulations based on these variably expressed genes.

(iii) Performing a Dimension Reduction Analysis, Preferably, a Principal Component Analysis for the Datasets of the Individual Cells Resulting in Clustering of the Individual Cells in Different Subpopulations Based on Features Derived from Said Datasets

From the previous step, all the genes with a high variance (typically v>0 with a Fractal dimensionality reduction <0.005) as an input for the dimensionality reduction are to be used. The main linear technique for dimensionality reduction is the Principal Component Analysis (PCA). PCA performs a linear mapping of the data to a lower-dimensional space in such a way that the variance of the data in the low-dimensional representation is maximized. In a PCA plot, each cell is plotted. Cells plotted close to each other have a similar transcriptome. The length of distance corresponds to similarity. The longer the distance between two cells, the more un-similar they are. Another way to reduce the dimensions is to perform t-stochastic neighbour embedding (t-SNE) for the cells (nonlinear), based on the PCA dimensions. The matrix of existing reduced dimensions may be taken from the PCA. By default, all dimensions are used to compute the second set of reduced dimensions. Results can be plotted in order to create tSNE maps that demonstrate the different sub-clusters on a map. Selection of perplexity: “The value of the perplexity parameter can have a large effect on the results. By default, the function will set a “reasonable” perplexity that scales with the number of cells in x. However, it is often worthwhile to manually try multiple values to ensure that the conclusions are robust. This is a step that is usually performed later, after integrating all the datasets (coming from different sequencing plates) into one.

(iv) Integrating RNAseq Datasets from Different Sequencing Plates and Clustering B-Cells in Different Subpopulations with Integrative Non Negative Matrix Factorization

In a preferred embodiment of the aforementioned method of the manufacture of the antibody of the present invention, the cell, gene and dimension data based on various experiments (investigated plates) are merged into one dataset. The integration approach is based on integrative non-negative matrix factorization (iNMF) using rliger package (Welch 2019). Preferably, datasets of individual cells from different splenic samples and experiments may be integrated. Thereby, technical variabilities may be counterbalanced. Since single cells are sorted into different places of a plate, there may be some technical variability. After merging the data for all the cells coming from different sequencing plates, again gene normalization, gene selection based on the variance (>0.1) and scaling takes place using functions from the corresponding functions of the rliger package. Then, iNMF is performed with the “optimizeALS” function to integrate the datasets. iNMF results in the identification of factors (factor loadings—metagenes) for each cell of the new joint dataset. Each factor corresponds to a biological signal and can characterize specific subclusters. Joint clustering is typically performed after iNMF. Based on the factor loading a label is assigned in each cell of the dataset using the “quantile_norm” function, and a shared factor neiborbood graph is built where cells sharing a similar factor loading pattern are connected. At the same step quantile normalization is performed to normalize the corresponding clusters, factors and datasets. The Louvain algorithm is applied to merge the smaller subclusters together.

(v) Identifying Individual Memory B-Cells as Cells from a Subpopulation of Cells Identified by Clustering

Using the function “runUMAP” on the normalized factors, we visualize the data graphically in two dimensions. UMAP (Uniform Manifold Approximation and Projection) is a novel manifold learning technique for dimension reduction and is included as a function of the rliger package. UMAP is constructed from a theoretical framework based in Riemannian geometry and algebraic topology. The result is a practical scalable algorithm that applies to real world data. A UMAP plot is a graph displaying the “Uniform Manifold Approximation and Projection”, which visually shows how separable the classes under consideration are with respect to the selected group of features. It is a 2D plot and represents each class as a cluster of points in a unique color. To identify the genetic markers, the runWilcoxon function of rLiger package was used. Alternatively, the MAST package form Finak 2015, Genome Biology 16(278) https://doi.org/10.1186/s13059-015-0844-5 available through the Seurat version 4.0 may be used.

In a preferred embodiment of the aforementioned method of the manufacture of the antibody of the present invention, said assembling antibody light and heavy variable chain encoding nucleic acid sequences from the nucleic acid sequence of the plurality of expressed genes of the selected individual memory B-cells is carried out by assembling a VDJ contig sequence based on the determined nucleic acid sequences encoding the antibody heavy and light chains comprised in the plurality of expressed genes and a reference database containing pre-complied variable heavy chain, constant heavy chain, variable light chain, and constant light chain sequences using a comparison algorithm for assembling the contig sequence. More preferably, said comparison algorithm and reference database is the BASIC algorithm and database. The BASIC software uses a pre-compiled database of known variable (IGHV, IGKV, IGLV) and constant (IGHC, IGKC, IGLC) region sequences in human from IMGT (http://www.imgt.org). The database was indexed using Bowtie2 into four distinct files that corresponded to different components (IGHV; variable heavy chain, IGHC; constant heavy chain, IGK/LV; variable light chain, IGK/LC; constant light chain). This is basically a dedicated alignment tool that is optimized for antibody sequence assembly. BASIC calls Bowtie2 (Version 2.2.5) in order to align scRNA-seq reads from a single B cell to each of the four component index files. “For each component, BASIC identifies a short sequence window containing the highest number of aligned reads. These four sequences served as anchors to guide the assembly stage. BASIC performs de novo assembly to stitch together the anchor sequences in the heavy and light chains. It can be assumed that a sequence may overlap either with the forward sequence or with the reverse complement sequence of another read. Two reads overlap if the prefix of one sequence equals the suffix of the other sequence or vice versa. BASIC extends each anchor iteratively in the 3′ direction (one read at a time) until there is either no overlapping read or a repeat is found. Then, each anchor is extended in the 5′ direction in the same way. For each chain, BASIC reports a single sequence if the extended sequence from the variable region anchor is equal to the extended sequence from the constant region anchor. At this point, the VDJ contigs of the variable antibody region have been assembled. To annotate the assembled VDJ contigs, the IgBLAST (v1.16.0) may be used. Using Blast on the the filtered and trimmed fastq files (output of step 1.1), the constant regions may be identified that identify the Ig Isotype (IgG, IgM, IgA, etc.). Sequenced antibodies may be incomplete in the context of recombinant expression. For example, the IDs of the V, D, and J segments of the vast majority of the sequences can be easily identified even if there are some “missing” segments in the sequence. In order to remove non-functional antibodies, a filtration step was used that aims at removing antibody sequences due to, for example, a frameshift mutation somewhere in the sequence, or a truncation at one end of the VDJ. Moreover, only antibodies for which both the VDJ of the heavy and light chains are identified and functional are of interest. Cells with sequence data for only one chain can be removed as well. In case the tool identifies 2 contigs in the same cell, with the same percentage of confidence, the contig the identification of which was based on the alignment of the longer sequence can be selected. (Example: antibody gene families are so expanded that a 20 bp fragment is likely to align perfectly with a number of different gene IDs. However, in one instance of this, the alignment to gene A might only be 17 bp worth, while the alignment to gene B might be 19 bp long. Therefore, it can be assumed that the fragment aligns to gene B). Only the VDJ sequences that belong to cells that showed good quality scores during the transcriptomic analysis were considered.

In a preferred embodiment of the aforementioned method of the manufacture of the antibody of the present invention, at least steps c), d) and/or e) are carried out by a data processing device. Preferably, a data processing device as referred to herein is configured by tangibly embedding the computer program-based algorithm for carrying out the method of the invention on said device at least partially. The processing device may comprise at least one integrated circuit configured for performing logical operations. Typically, the processor may also comprise at least one application-specific integrated circuit (ASIC) and/or at least one field-programmable gate array (FPGA). Moreover, the data processing device may comprise a memory for storing data functionally connected thereto. The memory may be a permanently or temporarily connected, physical data storage device. Preferably, the data processing device is a computer.

In a preferred embodiment of the aforementioned method of the manufacture of the antibody of the present invention, said expressing the antibody light and heavy chain encoding nucleic acid sequences assembled in step e) in a host cell in order to manufacture the antibody comprises:
(i) generating an expression plasmid for the antibody light and heavy chain;
(ii) introducing said expression plasmid into the host cell and allowing for expression of the antibody.

In a preferred embodiment of the aforementioned method of the manufacture of the antibody of the present invention, said method further comprises isolating said antibody from the said host cell. Isolating the antibody from the host cell can be achieved by purifying or partially purifying the antibody from the host cells or host cell culture. For protein purification, various techniques may be used including precipitation, filtration, ultra-filtration, extraction, chromatography techniques such as ion-exchange-, affinity- and/or size exclusion chromatography, HPLC or electrophoresis. The skilled person is well aware of how an antibody may be purified in order to provide it in isolated form. Preferred techniques are those described in the accompanying Examples below.

In a preferred embodiment of the aforementioned method of the manufacture of the antibody of the present invention, said host cell is a bacterial cell, a fungal cell or a eukaryotic cell. Preferably, said eukaryotic cell is selected from the group consisting of: HEK293T cells, CHO cells, BHK cells, NSO-GS cells, and cell lines derived from any of the aforementioned cells. Cell culture conditions for the aforementioned host cells which allow for expression of the antibody are well known to the person skilled in the art.

The term “carfentanil” as used herein refers to the compound (4-Methoxycarbonyl)fentanyl (CAS number 59708-52-0). It is a synthetic derivative of fentanyl approved for veterinary use (Wildnil). Carfentanil is a mu-opioid receptor agonist with an estimated analgesic potency about 10,000 times higher than that of morphine and 100 times higher that of fentanyl, based on animal studies. The toxicity of carfentanil has been compared to that of nerve gas. Since 2016, an increasing number of reports describe detection of carfentanil in the illicit drug supply throughout the United States, Europe and Canada. Moreover, carfentanil has a high potential as a chemical weapon and has been reported to be used in the form of an aerosol mist to subdue terrorists (Moscow theatre hostage crisis in 2002).

The present invention further relates to an antibody which specifically binds to carfentanil. Preferably said antibody binds to carfentanil with an equilibrium dissociation constant (Kd) of at most 1.000 nM, at most 500 nM, at most 100 nM, at most 80 nM, at most 60 nM, at most 40 nM, at most 20 nM, at most 10 nM, at most 8 nM, at most 6 nM, at most 4 nM, at most 2 nM, at most 1 nM, at most 800 pM, at most 600 pM, at most 400 pM, at most 200 pM, or at most 100 pM. In a preferred embodiment, the binding of the antibody and carfentanil shall be with a Kd in the picomolar range. More preferably, the binding of the antibody and carfentanil shall be with a Kd of at most 100 pM. The equilibrium dissociation constant referred to in accordance with the present invention can be determined by techniques well known in the art and described herein before, preferably, it is to be determined using Octet described in the accompanying Examples, below.

In a preferred embodiment, the binding pocket for carfentanil comprises amino acids from all three complementary determining regions (CDRs) of each chain. In a more preferred embodiment, the antibody comprises a light chain having (a) an amino acid sequence of SEQ ID NO: 5, or (b) an amino acid sequence which differs by at least one amino acid exchange, deletion and/or addition from SEQ ID NO: 5. In a more preferred embodiment, the antibody comprises a heavy chain having (a) an amino acid sequence of SEQ ID NO: 19, or (b) an amino acid sequence which differs by at least one amino acid exchange, deletion and/or addition from SEQ ID NO: 19. In the most preferred embodiment, the antibody comprises a light chain having an amino acid sequence of SEQ ID NO: 5 and a heavy chain having an amino acid sequence of SEQ ID NO: 19.

Adverse carfentanil actions in mice as referred to in this context of the present invention are, preferably, an impairment of nociception. More preferably, impairment of nociception can be tested by applying a hot stimulus to mice that have received carfentanil. Mice that are pre-treated by the antibody of the invention are protected from the adverse carfentanil actions and will exhibit restored or partially restored nociception while carfentanil treated mice that are not pre-treated by the antibody will exhibit impaired nociception caused by carfentanil. Impaired nociception is typically measured by determining the latency of a reaction of a mouse upon applying a hot temperature stimulus, e.g., using the hot plate test. Suitable test for measuring nociception are also well known in the art. Protection as referred to in this context of the present invention means that mice which received the antibody of the invention are either fully or partially protected from those adverse carfentanil actions, i.e. they shall react essentially as control mice that have not received fentanyl. The protection may be full protection or partial protection to a statistically significant extent. The dosage to be administered to mice can be provided via conventional routes of administration such as injection intra peritoneal. Preferably, the dosage is administered once prior to the administration of carfentanil. Typically, there is a time window of about 24 h between the administration of the antibody of the invention and the administration of carfentanil. Preferably, carfentanil is administered in dosages of about 0.01, 0.05 or 0.15 mg/kg.

Advantageously, it has been found in accordance with the studies underlying the present invention that antibodies can be generated which are capable of binding to carfentanil with a particular high affinity with Kds in the nano-molar or even pico-molar range. These antibodies allow for neutralizing carfentanil and, thus, for preventing or treating its adverse effects even if administered at rather low dosage. Using the technology for developing antibodies that specifically bind to carfentanil with particular high affinity, typically, with an affinity in the nano-molar or pico-molar range, and which require low dosage for eliciting protection.

Thanks to the present invention, therapeutic carfentanil use can be improved since its adverse side effects can be treated or prevented. Moreover, carfentanil abuse can be treated and/or prevented as well.

In addition, the present invention relates to a method of preventing and mitigating toxic effects of carfentanil. Preferably, the present invention can be used to prevent environmental toxicity, e.g. toxicity associated with carfentanil being introduced to the environment in the form of aerosol mist (e.g. carfentanil used as an air-borne chemical weapon such as during the Moscow theatre hostage crisis in 2002).

The present invention may also be used in a method of detecting carfentanil in a sample. Preferably, the aforementioned method of the present invention is used in a diagnostic setting or military defense setting. Thus, the sample to be used in said method is, preferably, a sample of a subject, such as a body fluid sample or tissue sample, or an environmental sample such as a soil, air or liquid sample from the environment. More preferably, the present invention is used in environmental diagnostics.

The present invention also refers to a kit for detecting carfentanil in a sample, said kit comprising (a) a container comprising an antibody as defined by the present invention, and (b) instructions for using the antibody for the purpose of detecting carfentanil in a sample.

The following are particular preferred embodiments of the present invention.

Embodiment 1: An antibody which specifically binds to a hapten being fentanyl or a derivative thereof, said antibody binding to the hapten with an equilibrium dissociation constant (Kd) of at most 1.000 pM, at most 800 pM, at most 600 pM, at most 400 pM, at most 200 pM, at most 100 pM or at most 75 pM, wherein the binding pocket for the hapten comprises amino acids from all three complementary determining regions (CDRs) of each chain and wherein said antibody is capable of protecting mice from adverse fentanyl actions when administered at a dosage (antibody compound (mg)/mouse body weight (kg)) of at most 10 mg/kg, at most 8 mg/kg, at most 6 mg/kg, at most 5 mg/kg, at most 4 mg/kg at most 3 mg/kg at most 2 mg/kg or at most 1 mg/kg.

Embodiment 2: The antibody of embodiment 1, wherein said hapten is fentanyl.

Embodiment 3: The antibody of embodiment 1 or 2, wherein said antibody comprises at least one heavy chain CDR said heavy chain CDR being
(I) a heavy chain CDR1 having an amino acid sequence selected from the group consisting of:
   (a) an amino acid sequence as shown in any one of SEQ ID NOs: 32, 33, 34, 35, 36, or 37; and
   (b) an amino acid sequence which differs by at least one amino acid exchange, deletion and/or addition from the sequence as shown in any one of SEQ ID NOs: 32, 33, 34, 35, 36, or 37;
(II) a heavy chain CDR2 having an amino acid sequence selected from the group consisting of:
   (a) an amino acid sequence as shown in any one of SEQ ID NOs: 53, 54, 55, 56, 57, 58, or 59; and
   (b) an amino acid sequence which differs by at least one amino acid exchange, deletion and/or addition from the sequence as shown in any one of SEQ ID NOs: 53, 54, 56, 57, 58, or 59; or
(III) a heavy chain CDR3 having an amino acid sequence selected from the group consisting of:
   (a) an amino acid sequence as shown in any one of SEQ ID NOs: 81, 82, 83, 84, 85, 86, 87, 89, or 90; and
   (b) an amino acid sequence which differs by at least one amino acid exchange, deletion and/or addition from the sequence as shown in any one of SEQ ID NOs: 81, 82, 83, 84, 85, 86, 87, 89, or 90.

Embodiment 4. The antibody of any one of embodiments 1 to 3, wherein said antibody comprises at least one light chain CDR, said light chain CDR being
(I) a light chain CDR1 having an amino acid sequence selected from the group consisting of:
   (a) an amino acid sequence as shown in any one of SEQ ID NOs: 38, 39, 40, or 41; and
   (b) an amino acid sequence which differs by at least one amino acid exchange, deletion and/or addition from the sequence as shown in any one of SEQ ID NOs: 38, 39, 40, or 41;
(II) a light chain CDR2 having an amino acid sequence selected from the group consisting of:
   (a) an amino acid sequence as shown in any one of SEQ ID NOs: 60, 61, or 62; and
   (b) an amino acid sequence which differs by at least one amino acid exchange, deletion and/or addition from the sequence as shown in any one of SEQ ID NOs: 60, 61, or 62; or
(III) a light chain CDR3 having an amino acid sequence selected from the group consisting of:
   (a) an amino acid sequence as shown in any one of SEQ ID NOs: 91, 92, 93, 94, 95, 96, 97, or 98; and
   (b) an amino acid sequence which differs by at least one amino acid exchange, deletion and/or addition from the sequence as shown in any one of SEQ ID NOs: 91, 92, 93, 94, 95, 96, or 97.

Embodiment 5. The antibody of any one of embodiments 1 to 4, wherein said antibody comprises at least one heavy chain FR, said heavy chain FR being
(I) a heavy chain FR1 having an amino acid sequence selected from the group consisting of:
   (a) an amino acid sequence as shown in any one of SEQ ID NOs: 22, 23, 24, 25, 26, 27 or 28; and
   (b) an amino acid sequence which differs by at least one amino acid exchange, deletion and/or addition from the sequence as shown in any one of SEQ ID NOs: 22, 23, 24, 25, 26, 27 or 28;
(II) a heavy chain FR2 having an amino acid sequence selected from the group consisting of:
   (a) an amino acid sequence as shown in any one of SEQ ID NOs: 42, 43, 44, 45, 46, 47 or 48; and
   (b) an amino acid sequence which differs by at least one amino acid exchange, deletion and/or addition from the sequence as shown in any one of SEQ ID NOs: 42, 43, 44, 45, 46, 47 or 48;
(III) a heavy chain FR3 having an amino acid sequence selected from the group consisting of:
   (a) an amino acid sequence as shown in any one of SEQ ID NOs: 63, 64, 65, 66, 67, 68, 69, 70 or 71; and
   (b) an amino acid sequence which differs by at least one amino acid exchange, deletion and/or addition from the sequence as shown in any one of SEQ ID NOs: 63, 64, 65, 66, 67, 68, 69, 70 or 71; or
(IV) a heavy chain FR4 having an amino acid sequence selected from the group consisting of:
   (a) an amino acid sequence as shown in any one of SEQ ID NOs: 99, 100, 101 or 102; and
   (b) an amino acid sequence which differs by at least one amino acid exchange, deletion and/or addition from the sequence as shown in any one of SEQ ID NOs: 99, 100, 101 or 102.

Embodiment 6. The antibody of any one of embodiments 1 to 5, wherein said antibody comprises at least one light chain FR, said light chain FR being
(I) a light chain FR1 having an amino acid sequence selected from the group consisting of:
   (a) an amino acid sequence as shown in any one of SEQ ID NOs: 29, 30 or 31; and
   (b) an amino acid sequence which differs by at least one amino acid exchange, deletion and/or addition from the sequence as shown in any one of SEQ ID NOs: 29, 30 or 31;
(II) a light chain FR2 having an amino acid sequence selected from the group consisting of:
   (a) an amino acid sequence as shown in any one of SEQ ID NOs: 49, 50, 51 or 52; and
   (b) an amino acid sequence which differs by at least one amino acid exchange, deletion and/or addition from the sequence as shown in any one of SEQ ID NOs: 49, 50, 51 or 52.
(III) a light chain FR3 having an amino acid sequence selected from the group consisting of:
   (a) an amino acid sequence as shown in any one of SEQ ID NOs: 72, 73, 74, 75, 76, 77, 78, 79 or 80; and
   (b) an amino acid sequence which differs by at least one amino acid exchange, deletion and/or addition from the sequence as shown in any one of SEQ ID NOs: 72, 73, 74, 75, 76, 77, 78, 79 or 80; or
(IV) a light chain FR4 having an amino acid sequence selected from the group consisting of:
   (a) an amino acid sequence as shown in any one of SEQ ID NOs: 103, 104, 105 or 106; and
   (b) an amino acid sequence which differs by at least one amino acid exchange, deletion and/or addition from the sequence as shown in any one of SEQ ID NOs: 103, 104, 105 or 106.

Embodiment 7. The antibody of any one of embodiments 1 to 6, wherein said hapten binding pocket comprises at least the following amino acids, preferably, as shown in FIGS. 10A-10D:
(i) Trp 47, Tyr 35, Leu 96, Trp 110, Tyr 36, Ile 98, Tyr 91, Thr 106, Asp 108, Tyr 55, Tyr 49, Tyr 101, Gln 89, and Glu 99;
(ii) Val 37, His 35, Tyr 6, Ala 97, Tyr 96, Phe 98, Asp 104, Ile 98, Tyr 55, Tyr 102, Glu 98, Glu 99, Gly 101, Tyr 91, and Tyr 49
(iii) Val 37, Ala 97, His 35, asp 108, Phe 98, Met 98, Tyr 36, Tyr 96, Glu 99, Tyr 55, Tyr 91, Tyr 106, and Gln 89; or
(iv) Trp 110, Phe 98, Ala 97, His 35, Tyr 55, Ile 98, Tyr 96, Tyr 49, Asp 108, Tyr 91, Asn 32, Gln 89, and Glu 99.

Embodiment 8. The antibody of any one of embodiments 1 to 7, wherein said antibody comprises a heavy chain having an amino acid sequence selected from the group consisting of:
(a) an amino acid sequence as shown in any one of SEQ ID NOs: 1, 2, 3, 4, 5, 6, 7, 8, 9 or 10; and
(b) an amino acid sequence which differs by at least one amino acid exchange, deletion and/or addition from the sequence as shown in any one of SEQ ID NOs: 1, 2, 3, 4, 5, 6, 7, 8, 9 or 10.

Embodiment 9. The antibody of any one of embodiments 1 to 8, wherein said antibody comprises a light chain having an amino acid sequence selected from the group consisting of:
(a) an amino acid sequence as shown in any one of SEQ ID NOs: 11, 12, 13, 14, 15, 16, 17, 18, 19, 20 or 21; and
(b) an amino acid sequence which differs by at least one amino acid exchange, deletion and/or addition from the sequence as shown in any one of SEQ ID NOs: 11, 12, 13, 14, 15, 16, 17, 18, 19, 20 or 21.

Embodiment 10. The antibody of any one of embodiments 1 to 9, wherein said antibody does not bind specifically to naloxone and/or tramadol.

Embodiment 11. A polynucleotide encoding the antibody of any one of embodiments 1 to 10.

Embodiment 12. The polynucleotide of embodiment 11, wherein said polynucleotide is RNA or DNA.

Embodiment 13. A vector or expression construct comprising the polynucleotide of embodiment 12.

Embodiment 14. A host cell comprising the polynucleotide of embodiment 11 or 12 or the vector or expression construct of embodiment 13.

Embodiment 15. The host cell of embodiment 14, wherein said host cell is a bacterial cell, a fungal cell, an animal cell or a plant cell.

Embodiment 16. A non-human transgenic organism comprising the polynucleotide of embodiment 11 or 12 or the vector or expression construct of embodiment 13.

Embodiment 17. The non-human transgenic organism of embodiment 16, wherein said organism is an animal or a plant.

Embodiment 18. An antibody as defined in any one of embodiments 1 to 10 or a polynucleotide as defined in embodiment 11 or 12 for use in treating and/or preventing a disease or condition in a subject associated with administration fentanyl or a derivative thereof.

Embodiment 19. A method for the manufacture of the antibody of any one of embodiments 1 to 10 which specifically binds to a hapten being fentanyl or a derivative thereof, comprising the steps of:
a) contacting a splenic sample of an animal, preferably a mouse, which has been immunized with the hapten with labeled hapten;
b) isolating individual cells from that sample that:
   are CD19 positive;
   are CD138 negative;
   having bound the labeled hapten;
c) determining the nucleic acid sequences of a plurality of expressed genes, preferably, the entire transcriptome for each of said isolated individual cells;
d) selecting individual memory B-cells among the individual isolated cells by identifying the presence of nucleic acid sequences of one or more expressed genes selected from the group consisting of: BhIhe41, Parm1, CD80, CobI, IgG1, IgG2A, IgG2B, IgG3, IgG4, IgA, IgE, Sspn, Ackr2, Nt5e, and Mki67 within the nucleic acid sequences of a plurality of expressed genes;
e) assembling antibody light and heavy variable chain encoding nucleic acid sequences from the nucleic acid sequence of the plurality of expressed genes of the selected individual memory B-cells; and
f) expressing the antibody light and heavy chain encoding nucleic acid sequences assembled in step e) in a host cell in order to manufacture the antibody.

Embodiment 20. The method of embodiment 19, wherein said animal has been immunized with the hapten using an immunization method comprising the steps of:
(i) administering at least once an immunogenic particle exhibiting a plurality of hapten molecules as a priming step; and
(ii) administering at least once carrier molecules exhibiting single hapten molecules as a boosting step.

Embodiment 21. The method of embodiment 19 or 20, wherein said isolating individual cells in step b) is carried out by using single cell sorting techniques.

Embodiment 22. The method of any one of embodiments 19 to 21, wherein said isolating individual cells in step b) further comprises isolating individual cells that are viable cells.

Embodiment 23. The method of embodiment 22, wherein a viable cell is negative for 7-aminoactinomycin (7AAD) staining.

Embodiment 24. The method of any one of embodiments 19 to 23, wherein said method further comprises determining whether the isolated cells in step b) exhibit IgG on its surface.

Embodiment 25. The method of any one of embodiments 19 to 24, wherein said determining the nucleic acid sequences of a plurality of expressed genes in step c) is carried out by using a single cell sequencing technique.

Embodiment 26. The method of any one of embodiments 19 to 25, wherein said determining the nucleic acid sequences of a plurality of expressed genes in step c) comprises a bioinformatic evaluation of the determined nucleic acid sequences.

Embodiment 27. The method of embodiment 26, wherein said bioinformatic evaluation comprises generating datasets for each individual cell which contain data reflecting the in vivo expression profile.

Embodiment 28. The method of embodiment 27, wherein said generating datasets for each individual cell which contain data reflecting the expression profile comprises the steps of:
(i) removing low quality sequence reads;
(ii) removing adaptor sequences from carrying out single cell sequencing;
(iii) aligning nucleic acid sequences to an indexed reference genome;
(iv) compiling a BAM file for the plurality of nucleic acid sequences of expressed genes aligned to a reference genome for each individual cell, and allocating a quality score for the quality of the alignment of sequences to the indexed reference genome to each BAM file;
(v) removing low quality sequence reads based on the allocated quality score, preferably, Phred score;
(vi) annotating the aligned sequences of a BAM file to chromosomal loci;
(vii) compiling a count matrix dataset comprising identifier for the individual cells and identifier for the expressed genes;
(viii) removing low quality matrix datasets (representing individual cells) based on the following criteria: high percentage of mitochondrial genes, total number of nucleic acid sequence reads and number of expressed genes in an individual cell; and
(ix) removing individual genes that are significantly underrepresented;

Embodiment 29. The method of any one of embodiments 26 to 28, wherein said bioinformatic evaluation comprises cluster analysis of the individual cells based on the datasets for each individual cell which contain data reflecting the expression profile.

Embodiment 30. The method of embodiment 29, wherein said cluster analysis comprises the steps of:
(i) normalizing the expression levels for each gene between the datasets of the individual cells by deconvolution;
(ii) identifying the genes based on which the clustering will be made by modelling the variability of the gene expression of the expressed genes of a cell;
(iii) performing a dimension reduction analysis, preferably, a principal component analysis for the datasets of the individual cells resulting in clustering of the individual cells in different subpopulations based on features derived from said datasets;
(iv) integrating RNAseq datasets from different sequencing plates and clustering B-cells in different subpopulations with integrative non negative matrix factorization; and
(v) identifying individual memory B-cells as cells from a subpopulation of cells identified by clustering.

Embodiment 31. The method of any one of embodiments 19 to 30, wherein said assembling antibody light and heavy variable chain encoding nucleic acid sequences from the nucleic acid sequence of the plurality of expressed genes of the selected individual memory B-cells is carried out by assembling a VDJ contig sequence based on the determined nucleic acid sequences encoding the antibody heavy and light chains comprised in the plurality of expressed genes and a reference database containing pre-complied variable heavy chain, constant heavy chain, variable light chain, and constant light chain sequences using a comparison algorithm for assembling the contig sequence.

Embodiment 32. The method of embodiment 31, wherein said comparison algorithm and reference database is the BASIC algorithm and database.

Embodiment 33. The method of any one of embodiments 19 to 32, wherein at least steps c), d) and/or e) are carried out by a data processing device.

Embodiment 34. The method of any one of embodiments 19 to 33, wherein said expressing the antibody light and heavy chain encoding nucleic acid sequences assembled in step e) in a host cell in order to manufacture the antibody comprises:
(i) generating an expression plasmid for the antibody light and heavy chain;
(ii) introducing said expression plasmid into the host cell and allowing for expression of the antibody.

Embodiment 35. The method of embodiment 34, wherein said method further comprises isolating said antibody from the said host cell.

Embodiment 36. The method of embodiment 34 or 35, wherein said host cell is a bacterial cell, a fungal cell or a eukaryotic cell.

Embodiment 37. The method of embodiment 36, wherein said eukaryotic cell is selected from the group consisting of: HEK293T cells, CHO cells, BHK cells NSO-GS cells, and cell lines derived from any of the aforementioned cells.

Embodiment 38. An antibody which specifically binds to carfentanil, preferably said antibody binding to carfentanil with an equilibrium dissociation constant (Kd) of at most 1.000 nM, at most 500 nM, at most 100 nM, at most 80 nM, at most 60 nM, at most 40 nM, at most 20 nM, at most 10 nM, at most 8 nM, at most 6 nM, at most 4 nM, at most 2 nM, at most 1 nM, at most 800 pM, at most 600 pM, at most 400 pM, at most 200 pM, or at most 100 pM, wherein the binding pocket for carfentanil comprises amino acids from all three complementary determining regions (CDRs) of each chain.

Embodiment 39: The antibody of embodiment 38, wherein said antibody is capable of protecting mice from adverse carfentanil actions when administered at a dosage (antibody compound (mg)/mouse body weight (kg)) of at most 10 mg/kg, at most 8 mg/kg, at most 6 mg/kg, at most 5 mg/kg, at most 4 mg/kg at most 3 mg/kg at most 2 mg/kg or at most 1 mg/kg.

Embodiment 40: The antibody of embodiment 38 or 39, wherein said antibody comprises at least one heavy chain CDR said heavy chain CDR being
(I) a heavy chain CDR1 having an amino acid sequence selected from the group consisting of:
   (a) an amino acid sequence as shown in any one of SEQ ID NOs: 32, 33, 34, 35, 36, or 37; and
   (b) an amino acid sequence which differs by at least one amino acid exchange, deletion and/or addition from the sequence as shown in any one of SEQ ID NOs: 32, 33, 34, 35, 36, or 37;
(II) a heavy chain CDR2 having an amino acid sequence selected from the group consisting of:
   (a) an amino acid sequence as shown in any one of SEQ ID NOs: 53, 54, 55, 56, 57, 58, or 59; and
   (b) an amino acid sequence which differs by at least one amino acid exchange, deletion and/or addition from the sequence as shown in any one of SEQ ID NOs: 53, 54, 56, 57, 58, or 59; or
(III) a heavy chain CDR3 having an amino acid sequence selected from the group consisting of:
   (a) an amino acid sequence as shown in any one of SEQ ID NOs: 81, 82, 83, 84, 85, 86, 87, 89, or 90; and
   (b) an amino acid sequence which differs by at least one amino acid exchange, deletion and/or addition from the sequence as shown in any one of SEQ ID NOs: 81, 82, 83, 84, 85, 86, 87, 89, or 90.

Embodiment 41: The antibody of any one of embodiments 38 to 40, wherein said antibody comprises at least one light chain CDR, said light chain CDR being
(I) a light chain CDR1 having an amino acid sequence selected from the group consisting of:
   (a) an amino acid sequence as shown in any one of SEQ ID NOs: 38, 39, 40, or 41; and
   (b) an amino acid sequence which differs by at least one amino acid exchange, deletion and/or addition from the sequence as shown in any one of SEQ ID NOs: 38, 39, 40, or 41;
(II) a light chain CDR2 having an amino acid sequence selected from the group consisting of:
   (a) an amino acid sequence as shown in any one of SEQ ID NOs: 60, 61, or 62; and
   (b) an amino acid sequence which differs by at least one amino acid exchange, deletion and/or addition from the sequence as shown in any one of SEQ ID NOs: 60, 61, or 62; or
(III) a light chain CDR3 having an amino acid sequence selected from the group consisting of:
   (a) an amino acid sequence as shown in any one of SEQ ID NOs: 91, 92, 93, 94, 95, 96, 97, or 98; and
   (b) an amino acid sequence which differs by at least one amino acid exchange, deletion and/or addition from the sequence as shown in any one of SEQ ID NOs: 91, 92, 93, 94, 95, 96, or 97.

Embodiment 42. The antibody of any one of embodiments 38 to 41, wherein said antibody comprises at least one heavy chain FR, said heavy chain FR being
(I) a heavy chain FR1 having an amino acid sequence selected from the group consisting of:
   (a) an amino acid sequence as shown in any one of SEQ ID NOs: 22, 23, 24, 25, 26, 27 or 28; and
   (b) an amino acid sequence which differs by at least one amino acid exchange, deletion and/or addition from the sequence as shown in any one of SEQ ID NOs: 22, 23, 24, 25, 26, 27 or 28;
(II) a heavy chain FR2 having an amino acid sequence selected from the group consisting of:
   (a) an amino acid sequence as shown in any one of SEQ ID NOs: 42, 43, 44, 45, 46, 47 or 48; and
   (b) an amino acid sequence which differs by at least one amino acid exchange, deletion and/or addition from the sequence as shown in any one of SEQ ID NOs: 42, 43, 44, 45, 46, 47 or 48;
(III) a heavy chain FR3 having an amino acid sequence selected from the group consisting of:
   (a) an amino acid sequence as shown in any one of SEQ ID NOs: 63, 64, 65, 66, 67, 68, 69, 70 or 71; and
   (b) an amino acid sequence which differs by at least one amino acid exchange, deletion and/or addition from the sequence as shown in any one of SEQ ID NOs: 63, 64, 65, 66, 67, 68, 69, 70 or 71; or
(IV) a heavy chain FR4 having an amino acid sequence selected from the group consisting of:
   (a) an amino acid sequence as shown in any one of SEQ ID NOs: 99, 100, 101 or 102; and
   (b) an amino acid sequence which differs by at least one amino acid exchange, deletion and/or addition from the sequence as shown in any one of SEQ ID NOs: 99, 100, 101 or 102.

Embodiment 43. The antibody of any one of embodiments 38 to 42, wherein said antibody comprises at least one light chain FR, said light chain FR being
(I) a light chain FR1 having an amino acid sequence selected from the group consisting of:
   (a) an amino acid sequence as shown in any one of SEQ ID NOs: 29, 30 or 31; and
   (b) an amino acid sequence which differs by at least one amino acid exchange, deletion and/or addition from the sequence as shown in any one of SEQ ID NOs: 29, 30 or 31;
(II) a light chain FR2 having an amino acid sequence selected from the group consisting of:
   (a) an amino acid sequence as shown in any one of SEQ ID NOs: 49, 50, 51 or 52; and
   (b) an amino acid sequence which differs by at least one amino acid exchange, deletion and/or addition from the sequence as shown in any one of SEQ ID NOs: 49, 50, 51 or 52.
(III) a light chain FR3 having an amino acid sequence selected from the group consisting of:
   (a) an amino acid sequence as shown in any one of SEQ ID NOs: 72, 73, 74, 75, 76, 77, 78, 79 or 80; and
   (b) an amino acid sequence which differs by at least one amino acid exchange, deletion and/or addition from the sequence as shown in any one of SEQ ID NOs: 72, 73, 74, 75, 76, 77, 78, 79 or 80; or
(IV) a light chain FR4 having an amino acid sequence selected from the group consisting of:
   (a) an amino acid sequence as shown in any one of SEQ ID NOs: 103, 104, 105 or 106; and
   (b) an amino acid sequence which differs by at least one amino acid exchange, deletion and/or addition from the sequence as shown in any one of SEQ ID NOs: 103, 104, 105 or 106.

Embodiment 44. The antibody of any one of embodiments 38 to 43, wherein said antibody comprises a heavy chain having an amino acid sequence selected from the group consisting of:
(a) an amino acid sequence as shown in any one of SEQ ID NOs: 1, 2, 3, 4, 5, 6, 7, 9 or 10; and
(b) an amino acid sequence which differs by at least one amino acid exchange, deletion and/or addition from the sequence as shown in any one of SEQ ID NOs: 1, 2, 3, 4, 5, 6, 7, 9 or 10.

Embodiment 45. The antibody of any one of embodiments 38 to 44, wherein said antibody comprises a light chain having an amino acid sequence selected from the group consisting of:
(a) an amino acid sequence as shown in any one of SEQ ID NOs: 11, 12, 13, 14, 15, 16, 17, 19, 20 or 21; and
(b) an amino acid sequence which differs by at least one amino acid exchange, deletion and/or addition from the sequence as shown in any one of SEQ ID NOs: 11, 12, 13, 14, 15, 16, 17, 19, 20 or 21.

Embodiment 46. The antibody of any one of embodiments 38 to 45, wherein said antibody comprises a light chain having
(a) an amino acid sequence of SEQ ID NO: 5 or
(b) an amino acid sequence which differs by at least one amino acid exchange, deletion and/or addition from SEQ ID NO: 5.

Embodiment 47. The antibody of any one of embodiments 38 to 46, wherein said antibody comprises a heavy chain having
(a) an amino acid sequence of SEQ ID NO: 19 or
(b) an amino acid sequence which differs by at least one amino acid exchange, deletion and/or addition from SEQ ID NO: 19.

Embodiment 48. A polynucleotide encoding the antibody of any one of embodiments 38 to 47.

Embodiment 49. The polynucleotide of embodiment 48, wherein said polynucleotide is RNA or DNA.

Embodiment 50. A vector or expression construct comprising the polynucleotide of embodiment 48 or 49.

Embodiment 51. A host cell comprising the polynucleotide of embodiment 48 or 49 or the vector or expression construct of embodiment 50.

Embodiment 52. The host cell of embodiment 51, wherein said host cell is a bacterial cell, a fungal cell, an animal cell or a plant cell.

Embodiment 53. A non-human transgenic organism comprising the polynucleotide of embodiment 48 or 49 or the vector or expression construct of embodiment 50.

Embodiment 54. The non-human transgenic organism of embodiment 53, wherein said organism is an animal or a plant.

Embodiment 55. An antibody as defined in any one of embodiments 38 to 47 or a polynucleotide as defined in embodiment 48 or 49 for use in treating and/or preventing a disease or condition in a subject associated with administration carfentanil.

Embodiment 56. The antibody of embodiment 55, wherein the condition is environmental exposure to carfentanil.

Embodiment 57. A method of preventing or mitigating environmental toxicity associated with the administration of carfentanil, wherein the method comprises administering an effective amount of the antibody as defined in any one of embodiments 38 to 47.

Embodiment 58. The method of embodiment 57, wherein carfentanil is administered to the environment in the form of an aerosol mist.

Embodiment 59. Use of the antibody as defined in any one of embodiments 38 to 47 or the polynucleotide as defined in embodiments 48 or 49 in a method of detecting carfentanil in a sample.

Embodiment 60. An Antibody as defined in any one of embodiments 38 to 47 or a polynucleotide as defined in embodiment 48 or 49 for use in preventing adverse effects of carfentanil used as an environmental toxin.

Embodiment 61. Kit for detecting carfentanil in a sample, said kit comprising
(a) a container comprising an antibody as defined in any one of embodiments 38 to 47; and
(b) instructions for using the antibody for the purpose of detecting carfentanil in a sample.

All references cited throughout this specification are herewith incorporated by reference in their entirety as well as with respect to the specifically mentioned disclosure content.

### FIGURES

FIG. 1: Schematic description of the workflow used to identify optimal B cell receptors (antibodies) after vaccination. Spleens are harvested from vaccinated animals and the splenocytes are homogenized. B cells are identified through specific recognition of B cell surface proteins (e.g., CD19 or, as pictured; IgG) using labeling reagents. Antigen-binding B cells are identified through contacting using a fluorescently-labeled version of fentanyl hapten. Finally, single cells are sorted based on their identifications into 384-well plates for single-cell transcriptomic analyses.

FIG. 2: The marker genes detected for each of the six B cell clustered subpopulations in addition to several known B cell markers. Heatmap visualizing the log normalized z-scores of the top 10 statistically important gene-markers (rows) for each population (columns) resulting from univariate differential expression adjusted for covariates, with log fold change >1.5 and adjusted p-value <0.05. Several additional known B cell markers have been added to strengthen the annotation of the identified subpopulations. Darker shading indicates genes that are underrepresented in certain populations, while light shading indicates genes that are overrepresented in certain populations. Hierarchical clustering has been applied to both rows and columns to group together relevel genes and identify similarities between clusters based on their expression profile.

FIGS. 3A and 3B: The memory B cell compartment produces the highest affinity B cell receptors. FIG. 3A. UMAP plot that shows the output of the clustering algorithms applied to the data represented by FIG. 2. The B cells form distinct clusters, with the most separated clusters (and thus the most well-characterized) being the GC-LZ-PrePB cells (bottom left) and the Switched-Memory cells (far right). The boxed numbers (e.g., 457208_1) indicate the individual cells from which antibody sequences were chosen for characterization. FIG. 3B. Flow cytometry data (Y axis: log 10 of the IgG1 surface expression level measured by the mean fluorescence intensity --- X axis: log 10 of the fluorescent fentanyl-binding capacity via contacting) obtained during the penultimate step of FIG. 1 replotted here to incorporate the output of the clustering algorithms. Different grey scales describe the different subclusters. The cells present in the upper and right-shifted population represent the cells that have the highest fentanyl-affinity and have “switched” to IgG1 expression (through class-switch recombination). This population is almost entirely represented by cells from the memory compartment.

FIG. 4: Schematic overview of the workflow used to produce the antibodies. Left: Antibody sequences are synthesized and cloned into expression vectors (Fabs gain a HIS-tag here). The antibodies are then transiently overexpressed in suspended HEK293F cells on a rotating shaking platform. Supernatants are collected and the antibodies are then purified according to the nature of the proteins as indicated. Right: A Coomassie-stained non-reducing SDS PAGE analysis of a series of purifications, revealing that the antibodies are successfully produced and are highly pure after purification. The (*) indicates a shifted band present in the Fab 609 lane; the heavy chain of this Fab is heterogeneously N-glycosylated, leading to the size-shift seen here.

FIG. 5: The antibodies do not cross react with non-fentanyl class opioids. Competition ELISA data obtained using mAb 136. The antibody was used to probe ELISA plates coated with fentanyl in the presence or absence of varied concentrations of binding inhibitors. Soluble fentanyl at higher concentrations inhibits binding to the plate as expected, while soluble tramadol and naloxone do not.

FIGS. 6A and 6B. Purification and Crystallography of Recombinant Antibody-Fentanyl Complexes FIG. 6A. Four panels are shown for each antibody-ligand complex (labeled) with the crystallization and cooling conditions: Size exclusion chromatograms for final material used in crystallization experiments with corresponding labels to indicate antibody and whether the ligand was fentanyl or the hapten version (elution volumes are shown along with absorbance units at 280 nm; Superdex 200 increase 10/300 GL column), cryo-cooled crystal mounted in loop at the synchrotron during data collection, sample diffraction, and 2Fo-Fc electron density map focused on the ligand contoured at 1 sigma from final refinements. FIG. 6B. An SDS gel stained with Coomassie blue shows the contents of the peak fractions (M indicated molecular weight markers with several of those bands shown on the left side of the gel in kD whereas the gel lanes are labeled above for the antibody number).

FIGS. 7A-7C. Structure of the Antibody-fentanyl Complex. FIG. 7A. Overall structure of a complex of a Fab (FenAb609, heavy chain in light grey, light chain in darker grey) with fentanyl (space filling depiction) shown as a ribbon diagram with the two-dimensional chemical structure of fentanyl on the left. The CDR1, CDR2, and CDR3 regions of the antibody are highlighted in very light grey, dark grey, and light grey respectively. The partially transparent molecular surface of the protein is shown overlaid on the structure. FIG. 7B. Illustration of the fentanyl binding pocket as thin slice through the molecular surface of the protein (in a gradient from white to dark grey to reflect increasing hydrophobicity of the surface, colored using the method of Eisenberg(Eisenberg, 1984, J. Mol. Biol. 179, 125-142). Fentanyl is shown as a stick model with atoms of carbon, nitrogen, and oxygen in gray, dark gray, and white, respectively. FIG. 7C. Contacts of the protein to fentanyl shown with side chains as stick models and the main chain as a ribbon diagram. Fentanyl colored as in FIG. 7B, and the residues of the heavy and light chains adopting the colors from FIG. 7A. The CDR regions are colored as in FIG. 7A as well. “HC” denotes heavy chain and “LC” denotes the light chain. Hydrogen bonds are shown as dashed gray lines between bonded atoms.

FIG. 8. Structure-based sequence annotated alignment of antibodies. Sequences of four fentanyl-binding Fab molecules where the alignment was generating by superimposing the crystal structures. The secondary structure of a representative Fab (FenAb609) is shown above the sequence, colored as per FIG. 7A, as are CDR regions. Disulfide bonds are shown as lines connecting cysteine residues. Major and minor contacts are indicated in dark gray and light gray, respectively. The framework regions are denoted as FR1, 2, 3, and 4.

FIG. 9. Fentanyl can adopt different conformations in different antibody pockets. Superposition of FenAb609 and FenAb208 crystal structures with the molecular surface of FenAb208 shown (heavy and light chain regions colored as FIG. 7A) along with the stick figures of fentanyl in both molecules to illustrate the differing conformation of the top aromatic ring in the FenAb208 structure, which possesses a different CDR3 conformation which opens the molecular surface for the alternative conformation of the drug.

FIGS. 10A-10D. Schematic illustration of molecular contacts between different antibodies and fentanyl. The ligand is shown as a ball-and-stick model and the antibody contacts abstractly as partial circles near the atoms with which they interact. “Lig” and 7V7 represent the hapten and fentanyl-only forms, respectively.

FIGS. 11A-11C. Passive therapy protects from fentanyl intoxication. FIG. 11A. Schematic of the experimental design, whereby mice were passively immunized prior to exposure to fentanyl.

FIG. 11B. Behavioral readouts of fentanyl toxicity. Upper graph: representative data from the “hotplate-test,” whereby animals are assessed for their ability to sense a heat stimulus. The control group reveals the maximum possible effect (MPE; Y axis) that can be identified by the experimenter, while the experimental antibody (mAb 136 in this case) injected groups show a markedly reduced effect. A 4.5-fold reduction in % MPE could be observed at a dosage of 3.75 mg/kg body weight, while a complete reduction to baseline was obtained using 26.25 mg/kg of mAb136. FIG. 11C. mAb (F9) and mAB (B5) from WO2020/247584 approach baseline or elicit a 2-3-fold reduction only at a dosage of 40 mg/kg. mAb136 is superior and requires less dosage.

FIG. 12. Passive therapy prior to fentanyl exposure traps the fentanyl in the serum. Mass spectrometry results obtained after subjecting fentanyl-exposed mouse serum (harvested after conclusion of the experiment in FIG. 11B) to fentanyl quantitations by this technique. The passively delivered antibody traps the fentanyl in the serum of the animal, which thus prevents brain invasion of the drug, thereby preventing toxicity. At 3.75 mg/kg, there is a 5 to 10-fold increase in fentanyl serum-retention, while at 26.25 mg/kg there is a 50-100-fold increase.

FIGS. 13A and 13B. Fentanyl is retained in the serum after passive therapy in a 1:1 molar ratio relative to the delivered antibody. FIG. 13A. The amount of antibody present in the serum at the time of the fentanyl challenge was analyzed by western blot and is represented here. The horizontal lines indicate the value that would have been reached if 100% of the injected antibody was indeed present in the serum at the time of challenge. FIG. 13B. The experimentally determined values from FIG. 13A were converted to molar values and plotted against the molar amount of fentanyl trapped in the serum (as determined in FIG. 12 after conversion). At both the high and low doses, there is a 1:1 molar ratio of antibody to fentanyl in the serum, suggesting that mAb 136 is functioning in this protection experiment at nearly optimal levels.

FIG. 14. The antibodies cross react with more highly-potent fentanyl derivatives. Competition ELISA data obtained using 8 of the mAbs. The antibodies were used to probe ELISA plates coated with fentanyl in the presence or absence of varied concentrations of binding inhibitors (opioid molecules). Soluble fentanyl at higher concentrations inhibits binding to the plate as expected, while soluble tramadol and naloxone do not. Additional fentanyl-like molecules are shown, revealing that the antibodies do bind to more potent-fentanyl derivative molecules such as Acetylfentanyl, Norfentanyl and Carfentanil. Carfentanil is particularly highlighted by the darker X-marked circles, given its public health importance.

### EXAMPLES

The invention will be illustrated by the following Examples. The Examples shall not be construed as limiting the scope of the invention.

### Example 1: Generation of Antibodies

The inventors have generated a series of monoclonal antibodies that bind to fentanyl with high-affinity (via a unique mode of interaction created by a deep binding pocket) and can be used to block fentanyl intoxication in vivo using notably low doses. Essentially, these antibodies can provide a “sponge” that soaks up any fentanyl if injected into an individual experiencing opioid toxicity, or if injected prophylactically. These mAbs thereby offer a new variety of overdose prevention/treatment therapeutic options that otherwise currently consist exclusively of naloxone.

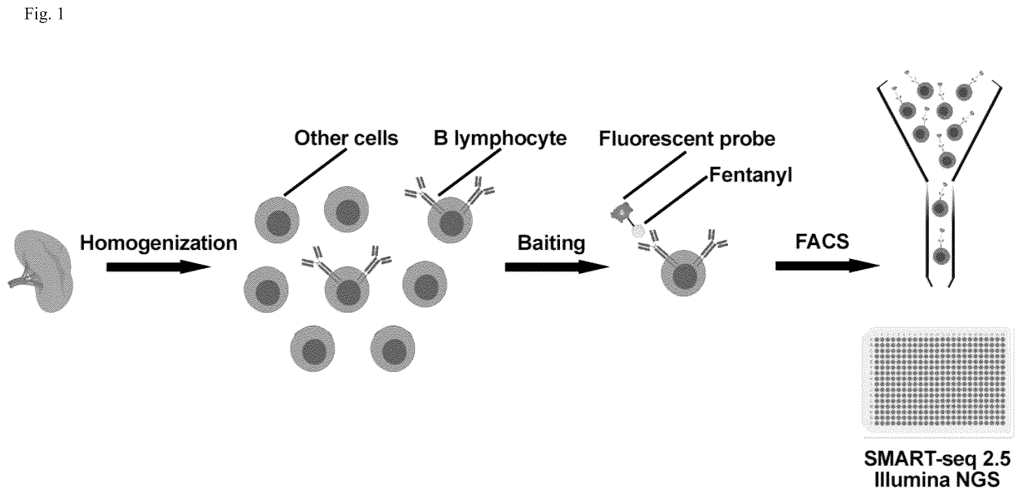

The antibodies were elicited by vaccination with an immunogenic fentanyl-presenting platform (see patent WO2020/084072A1). After vaccinating mice with several doses, the inventors harvest the spleens of the immunized animals. Spleens are homogenized in ice-cold PBS or RPMI medium by mashing, passed through a cell strainer, and pelleted. The pelleted splenocytes are resuspended in FCS with 10% DMSO for freezing. Upon the later thawing of the splenocytes, the cells are subjected to a fluorescent staining protocol that allows for the identification of hapten (fentanyl)binding B lymphocytes (characterized by the surface expression and detection of CD19, the absence of CD138, the ability to bind to fluorescently-labeled hapten, and the avoidance of the fluorescent decoy). The cells are then subjected to flow cytometry and single-cell sorting, which isolates individual antigen binding B cells in preparation for single-cell sequencing via the SmartSeq2 platform. The overall scheme of this process is described in FIG. 1.

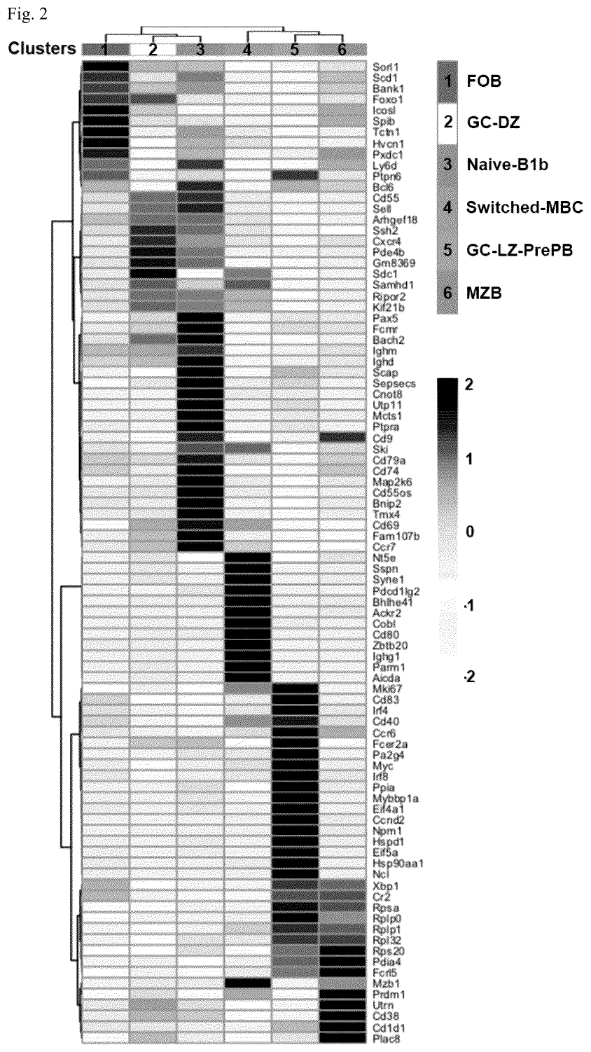

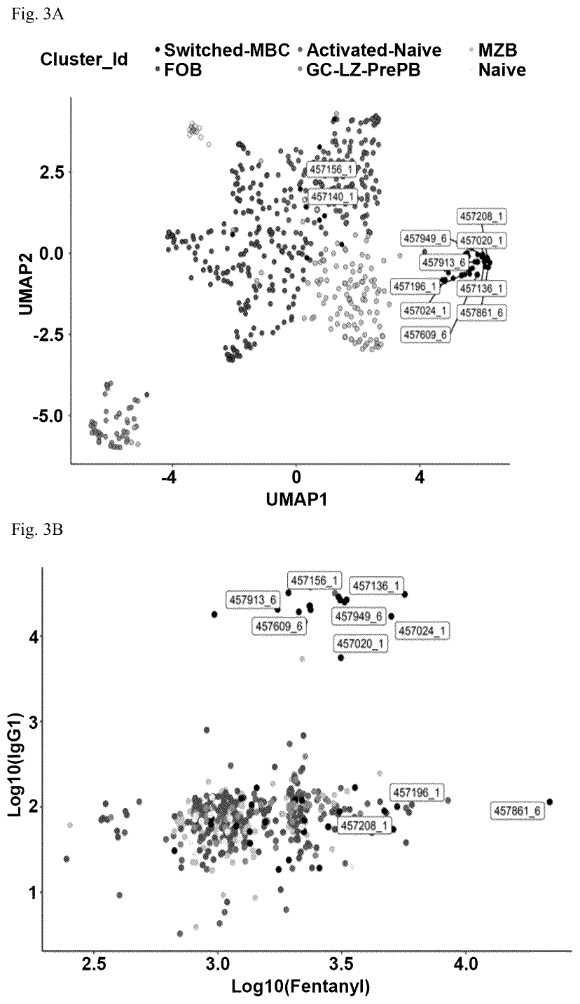

Memory B lymphocytes typically produce the most high-affinity IgG-class antibodies to any given hapten. The single-cell sequencing approach is designed to identify these memory cells by transcriptomic-level expression of “memory markers.” To do this, the transcriptomes are analyzed using a series of bioinformatic tools described elsewhere in this patent. Briefly, the genes that are differentially represented by each individual cell at a statistically relevant level are identified (FIG. 2 represents this data/concept). The expression levels of these genes (comprising a combination of known B cell subtype markers and markers originally identified throughout this work) in each cell are then used to cluster the cells by similarity to one another. In other words, 2 cells that have similar transcriptomic profiles will be assigned to a given cluster, while a 3^{rd} cell that has a number of differentially expressed genes relative to the first 2 cells may then represent and establish a 2′ cluster. The algorithms can be manually modified to assume the existence of a certain number of different clusters based on both visual and statistical confidence in the output. Here, the inventors have most confidently employed the expectation that 6 clusters exist in their B lymphocyte data sets, as shown in FIGS. 2 and 3A. The inventors then re-plot their flow cytometry data to show only the transcriptomically analyzed cells, determining that indeed the most high-affinity B lymphocytes (cells with the highest capacity to capture fentanyl) are indeed most generally from the memory cluster (FIG. 3B).

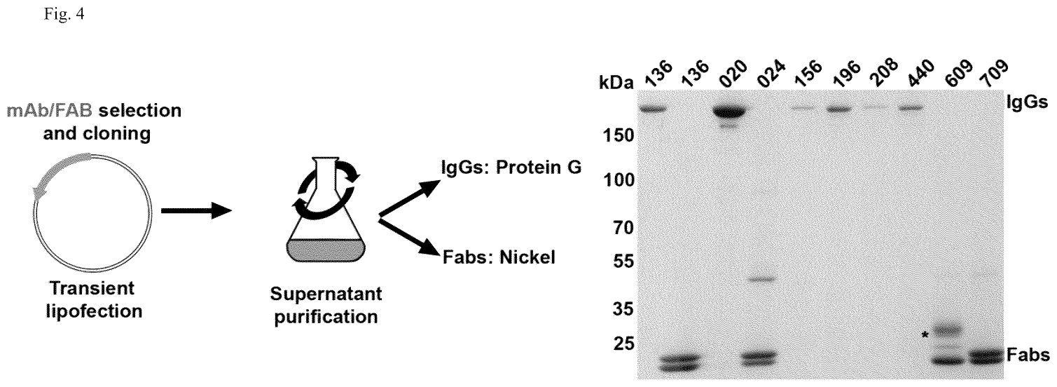

The presumed-to-be-high-affinity antibody sequences from the transcriptomically analyzed B lymphocytes were then synthesized and cloned into antibody expression vectors. For various reasons (elaborated below), the antibodies were cloned both as humanized IgG1 s (heretofore referred to simply as IgGs) and as murine antigen-binding fragments (Fabs). The Fab versions were cloned into and expressed from a vector that adds a HIS tag to the mature protein, while the IgGs were cloned into and expressed from the previously described humanized (encoding human constant regions) antibody vectors (Wardemann 2019, Methods Mol Biol 1956:105-125). The antibodies were expressed in HEK293F cells (grown in suspension with gentle agitation in serum-free FreeStyle 293 expression medium at 37 degrees Celcius with 5% CO2) after transient lipofection (using FreeStyle Max Reagent according to the manufacturers direction) of the antibody-encoding vectors (with the separate heavy and light chain-encoding plasmids being transfected in a 1:1 molar ratio to one another). The antibodies produced by these vectors encode signal peptides, and are therefore secreted into the supernatant for collection by the inventors. The antibodies were then purified out of the supernatants using one-step affinity-based purification strategies: Fabs (encoding HIS-tags) were bound to Nickel affinity resin and the contaminating unbound material was washed out (with 20 mM HEPES, 150 mM NaCl, and 10 mM imidazole at pH 7.4). Bound Fabs were then eluted (with 20 mM HEPES, 150 mM NaCl, and 300 mM imidazole at pH 7.4), concentrated, and dialyzed for subsequent analyses. IgGs were bound to Protein G resin and the contaminating unbound material was washed out (with 100 mM Tris, 150 mM NaCl, pH 7.4). IgGs were then eluted (with 100 mM glycine, 150 mM NaCl, pH 2.8 followed by an immediate neutralization with 1M Tris at pH 8.8), concentrated, and dialyzed for subsequent analyses. This work-flow is summarized in FIG. 4. 11 different antibodies were produced.

### Example 2: Structural and Functional Antibody Characterization

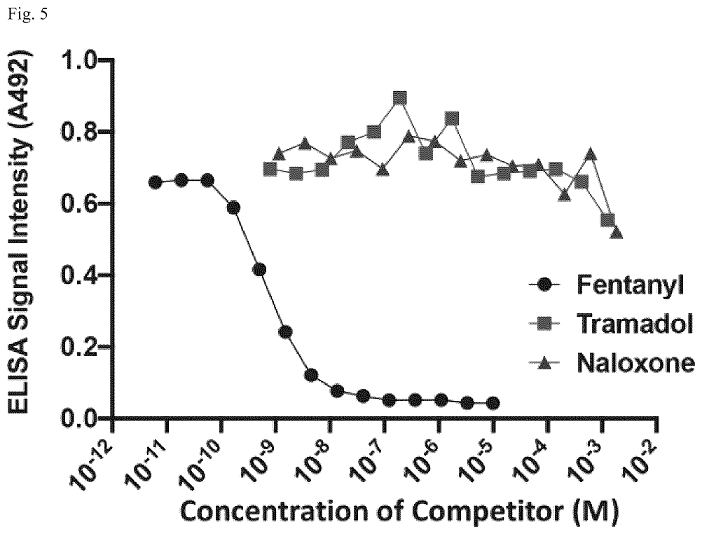

The 11 antibodies produced were subsequently characterized as follows. Antibodies were subjected to a series of ELISA assays to confirm that they bound to fentanyl, and to assess whether they bound to additional opioid molecules. The antibodies indeed can bind fentanyl, while they are unable to bind to the non-fentanyl class opioids Tramadol and Naloxone as determined by competition studies, whereby increasing amounts of soluble competitor are titrated into the ELISA. Representations of these data are shown in FIG. 5.

Binding affinities were determined by Octet (Biolayer Interferometry) using streptavidin-coated biosensors loaded with biotin-bound fentanyl hapten (loaded at 0.2 μg/mL for 60 seconds). Association and disassociation rates of the antibodies were measured in PBS/Tween at a variety of sample concentrations so as to confidently determine affinity constants. The affinities are reported in Table 1.

TABLE 1
Fentanyl-hapten binding affinities.

| Antibody | Kd | |
|---|---|---|
| 136 | 980 | pM |
| 709 | 916 | pM |
| 156 | 184 | pM |
| 913 | 132 | pM |
| 196 | 75 | pM |
| 024 | 74 | pM |
| 440 | 70 | pM |

| | |
|---|---|
| 020 | Too high to determine |
| 208 | Too high to determine |
| 609 | Too high to determine |
| 861 | Too high to determine |

The antibodies were subjected to octet assays using biotinylated fentanyl-hapten as the bait. The affinities reported here are well supported by the raw data except for those which are represented by the “too high to determine” value. These affinities were too high for the instrument and its algorithms to reliably call.

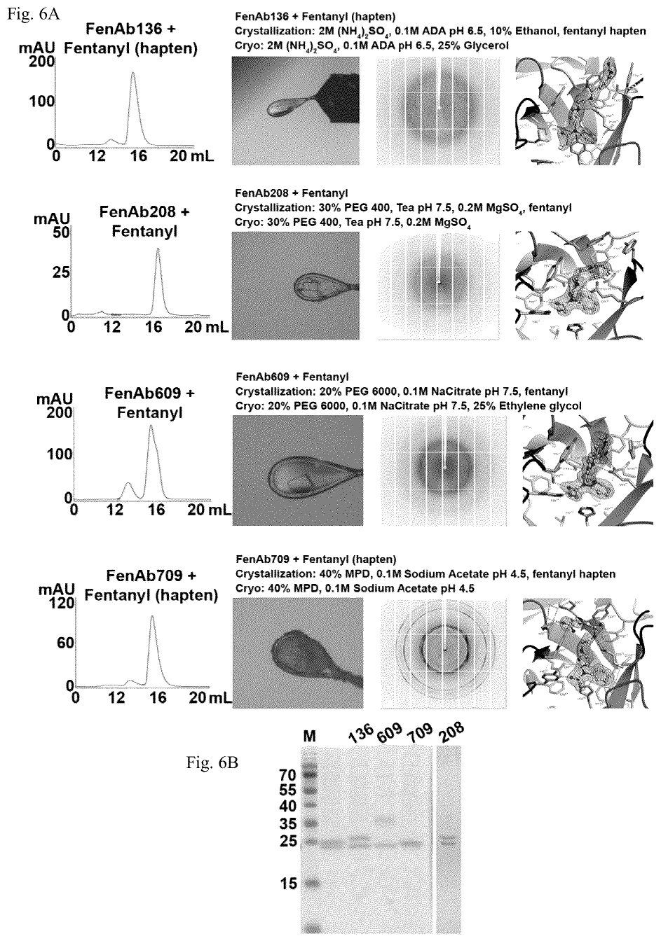

Fabs were analyzed by X-ray crystallography in order to determine the binding modality to fentanyl in molecular detail (IgGs are not suitable for these studies, while Fabs are easily crystallized). Fentanyl or fentanyl-hapten was bound to each Fab, prior to repurification by gel filtration. The complexes were then crystallized. The crystallization conditions and quality control analyses are shown in FIGS. 6A and 6B. All the datasets were collected at the Paul Scherrer Institut (Beam line X06DA) at a wavelength of 1.0 Å. For FenAb709 Mosflm was used to remove the ice rings. The structure of FenAb136 was solved by molecular replacement with PHENIX using 5H2B as a search model (Tatsumi 2017). All the other structures were solved using FenAb136 as a starting model. Autobuild was used for building the initial models. PHENIX, COOT and PDB-redo were used for building and refinement. The fentanyl or fentanyl-hapten was built after refinement on the protein structure and the density for the fentanyl was clearly visible.

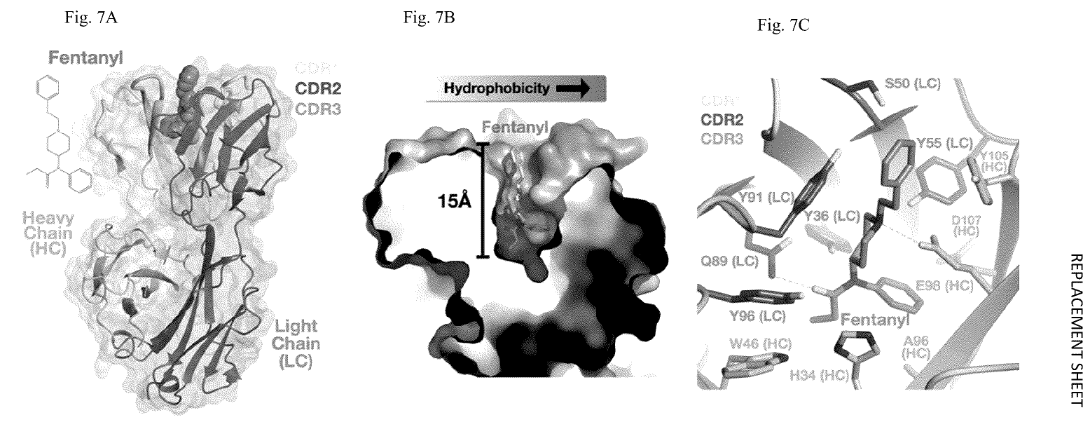

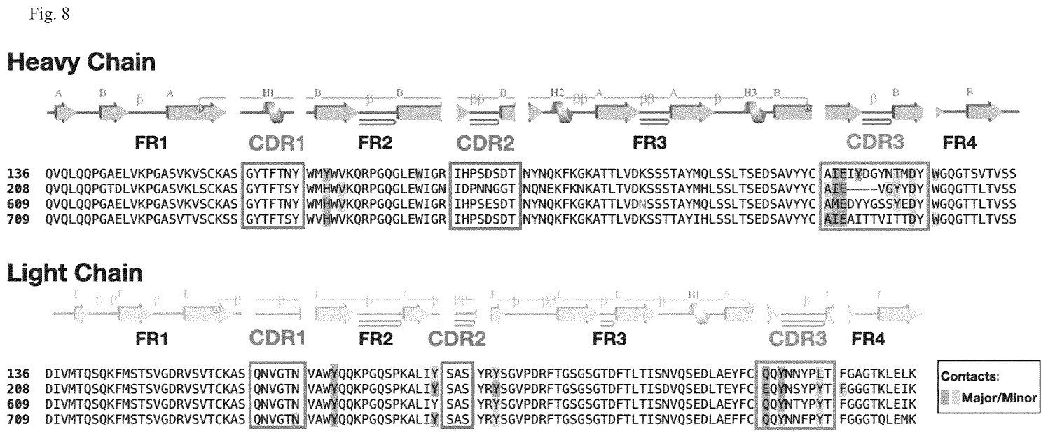

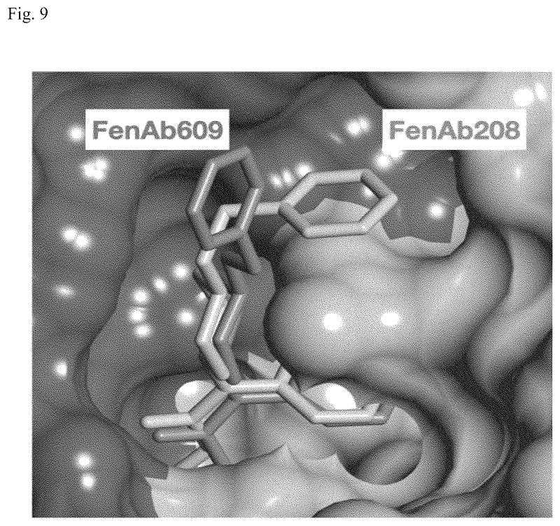

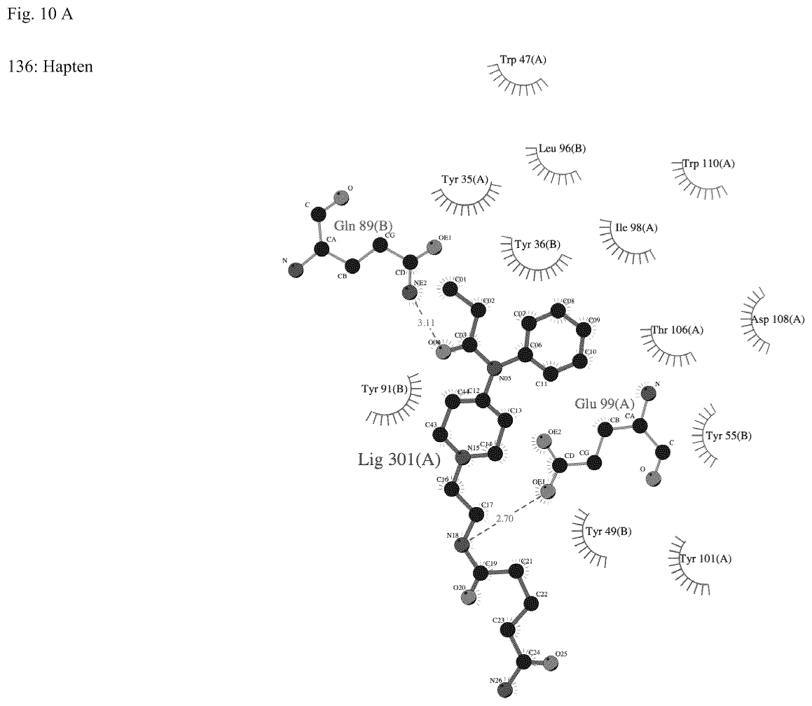

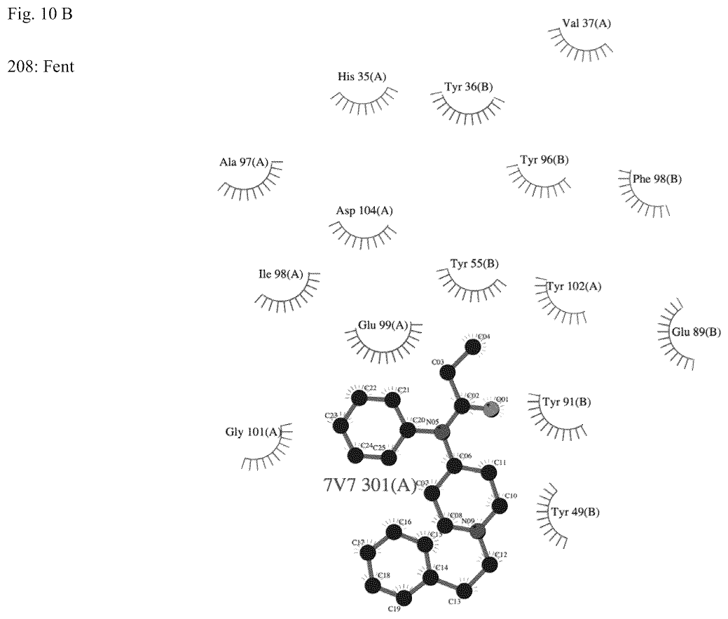

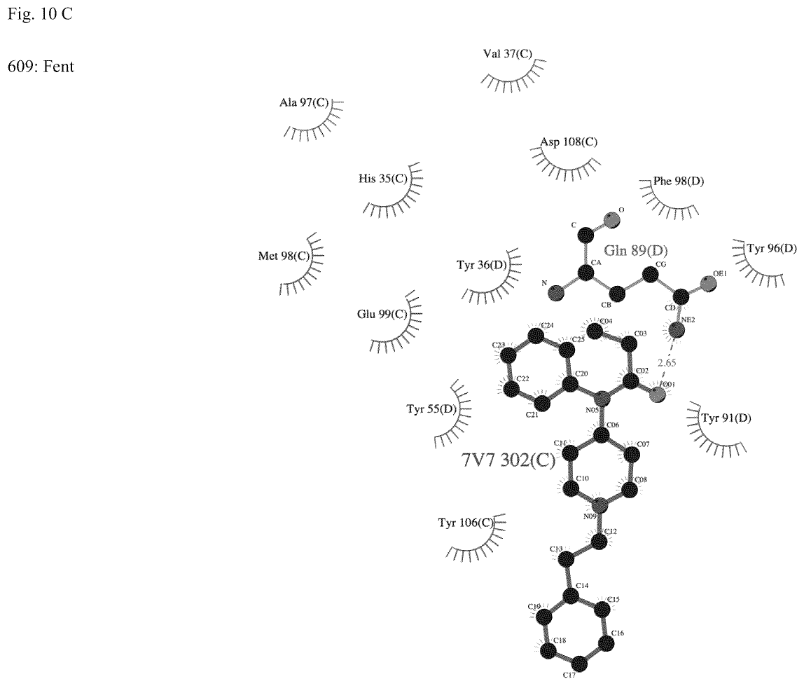

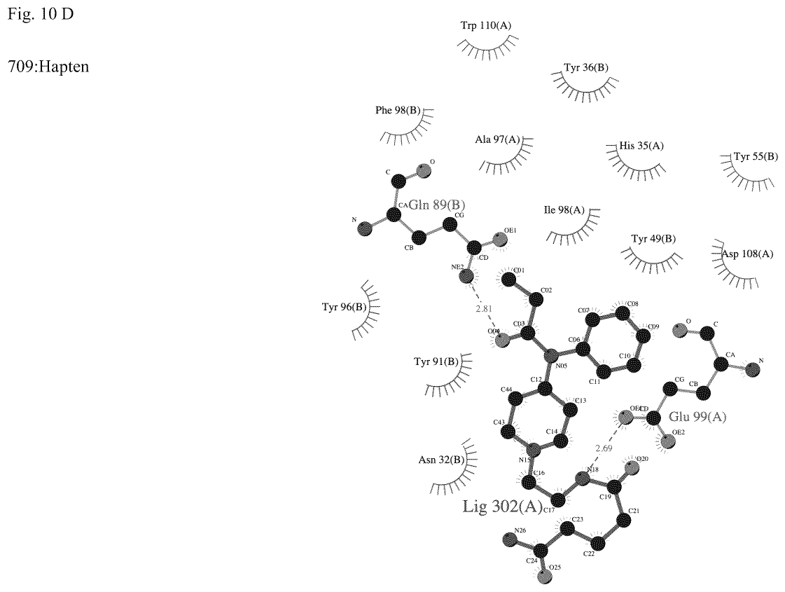

The crystallized Fabs reveal that the fentanyl molecules are captured by antibodies by a binding modality in the form of a “deep pocket.” FIGS. 7A and 7B reveal that the binding pocket is approximately 15 angstroms deep, which is critical to the binding mechanism as the fentanyl molecule becomes surrounded by the antibody. The major atomic contacts are depicted in FIG. 7C, which indeed coordinate the molecule from all directions, akin to an enzymatic active site or a “deep pocket.” All of the Fabs crystallized by the inventors while bound to fentanyl or fentanyl hapten form a similar deep pocket, which can also be visualized by their overall similarity at the sequence level (FIG. 8). However, interestingly the pocket is not identical between all antibodies, as the inventors show that the fentanyl molecule assumes multiple different binding conformers in the different structures, as represented in FIG. 9. Finally, the binding residues for all crystallized Fabs are shown in schematic form in FIGS. 10A-10D, highlighting both the similarities and differences between the different binding mechanisms utilized by each antibody.

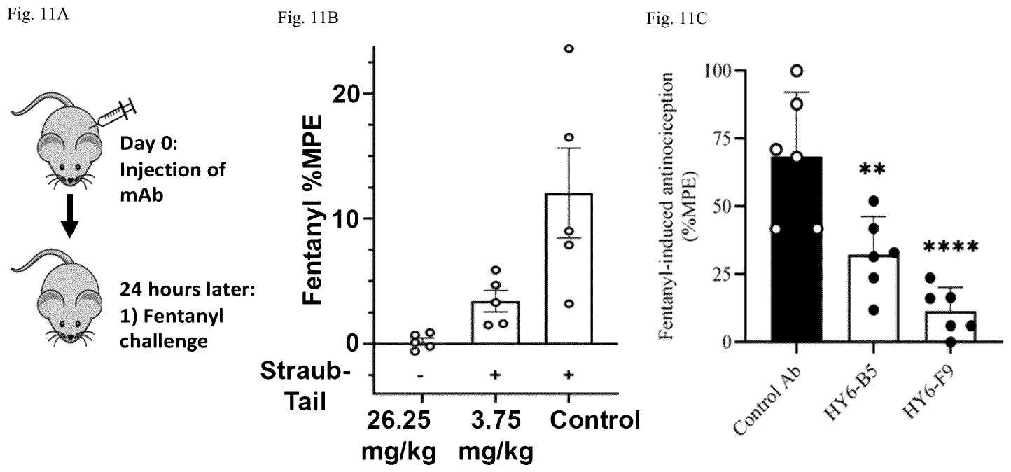

Some antibodies have been characterized for their ability to directly protect from fentanyl toxicity in mice. Specifically, IgG 136 was intraperitoneally injected into mice approximately 24 hours prior to exposure to fentanyl (FIG. 11A), after which the opioid-induced effects were assessed. Latency to respond to heat stimulation is a measure of analgesia, the results of which are shown in FIG. 11B where antibody injected mice show a marked reduction in observed maximum possible effect (% MPE). Importantly, mice injected with a dose of 26.25 mg/kg were completely unaffected by fentanyl toxicity, while mice injected with a low dose of only 3.75 mg/kg were partially protected (also exhibiting a minor but observable “Straub Tail response”). Recent reports of similar experiments have described significantly higher doses of antibody being needed in order to observe protective effects, thus establishing our antibody 136 (and likely many of its more high-affinity counterparts) as better candidates for human use.

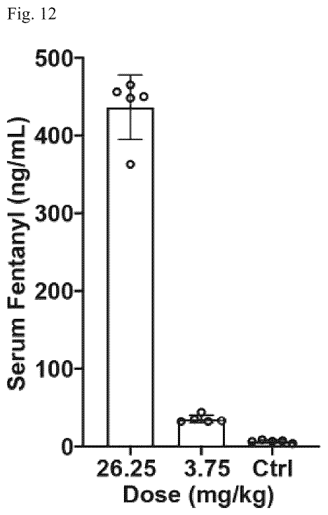

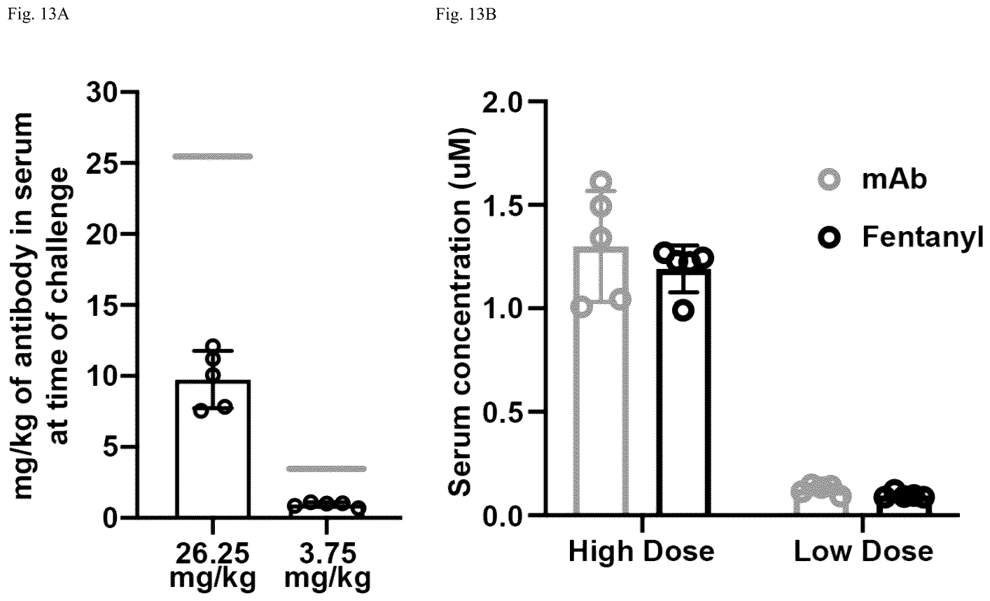

Serum was collected from the mice post-fentanyl challenge for a variety of assessments. Firstly, the amount of fentanyl retained in the serum was assessed by liquid chromatography-mass spectrometry (LCMS) on the basis that protective antibodies should bind to the fentanyl and prevent it from escaping the serum (and subsequently entering the brain). Indeed, LCMS revealed that control mice (not injected with a protective antibody) had little to no detectable serum-fentanyl, while the antibody injected mice had detectable serum-fentanyl amounts that were dependent on the amount of injected antibody (FIG. 12). The inventors also assessed the amount of actual IgG in the serum at the time of challenge by western blot, determining that value to be approximately 2.5-fold lower than the dose that was originally injected (less than 100% of the intraperitoneally injected mAb is expected to enter the bloodstream in general, although here it has been quantified explicitly) (FIG. 13A). The molar concentrations of serum-fentanyl and serum-IgG were determined be to nearly identical, indicating that nearly every antibody injected into the animal was capable of binding to a fentanyl molecule (FIG. 13B). These antibodies can thereby be effectively used as therapeutics in order to either treat or prevent opioid overdose.

### Example 3: Determination of Antibody Binding Affinities to Fentanyl and Fentanyl Derivatives

Binding affinities of the produced antibodies to fentanyl and fentanyl derivatives carfentanil, alfentanil, sufentanil, remifentanil, acetylfentanil and norfentanil were determined by a series of ELISA assays and measurement by Octet as described in Example 2.

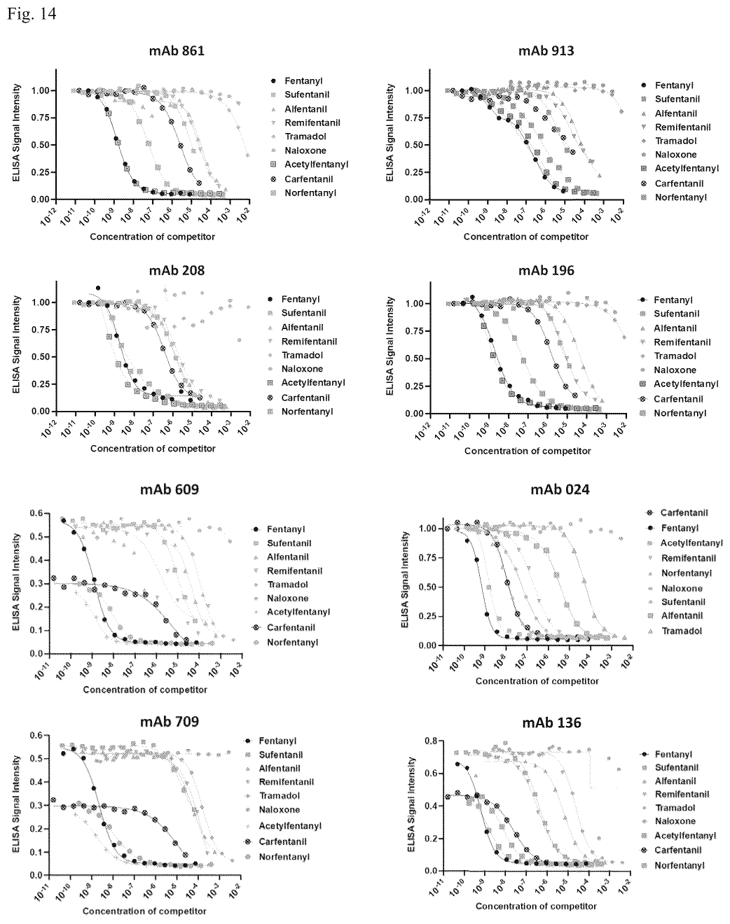

The antibodies demonstrated strong binding affinity to fentanyl and the fentanyl derivatives, while they are unable to bind the non-fentanyl class opioids tramadol and naloxone as determined by competition studies. Representations of these data are shown in FIG. 14.

Representative binding affinities for the fentanyl derivative carfentanil determined by Octet are reported in Table 2.

TABLE 2
Fentanyl and carfentanil binding affinities determined by Octet

| | | |
|---|---|---|
| Antibody ID | Fentanyl affinity | Carfentanil affinity |

| 136 | 980 | pM | 40 | nM |
|---|---|---|---|---|
| 913 | 132 | pM | 10 | nM |
| 196 | 75 | pM | 4 | nM |
| 709 | 916 | pM | >1 | uM |
| 208 | Too high to determine | | 95 | pM |
| 861 | Too high to determine | | 6 | nM |
| 024 | 74 | pM | 16 | nM |

| | | | |
|---|---|---|---|
| 609 | Too high to determine | >1 | uM |

The affinities reported here are well supported by the raw data except for those which are represented by the “too high to determine” value. These affinities were too high for the instrument and its algorithms to reliably call. Antibody mAb208 is of particular interest as it demonstrated a noticeably high affinity for carfentanil.

### CITED LITERATURE

Smith et al., 2019, J Am Chem Soc. 2019 Jul. 3; 141(26): 10489-10503;
Lefranc, et al., 2003, Dev Comp Immunol. 27(1): 55-77;
Triller 2017, Immunity 47(6): 1197-1209
Cho 2018, Nat. Commun. 9(1): 2757
Cho 2018, Nat. Comun. 16(9): 2757
Picelli 2014, Nature Protocols 9, 171-181
WO2021/214043
Lun 2016, Genome Biol. 17:75
Finak 2015, Genome Biology 16(278); https://doi.org/10.1186/s13059-015-0844-5
Eisenberg, 1984, J. Mol. Biol. 179, 125-142
Wardemann 2019, Methods Mol Biol 1956:105-125
Tatsumi 2017, Sci Rep 7: 43480-43480. DOI: 10.1038/srep43480
Canzar 2017, Bioinformatics, 33 (3): 425-227
Welch 2019, Cell 177(7): 1873-1887

1. An antibody which specifically binds to a hapten being fentanyl or a derivative thereof, said antibody binding to the hapten with an equilibrium dissociation constant (Kd) of at most 1.000 pM, at most 800 pM, at most 600 pM, at most 400 pM, at most 200 pM, at most 100 pM or at most 75 pM, wherein the binding pocket for the hapten comprises amino acids from all three complementary determining regions (CDRs) of each chain and wherein said antibody is capable of protecting mice from adverse fentanyl actions when administered at a dosage (antibody compound (mg)/mouse body weight (kg)) of at most 10 mg/kg, at most 8 mg/kg, at most 6 mg/kg, at most 5 mg/kg, at most 4 mg/kg at most 3 mg/kg at most 2 mg/kg or at most 1 mg/kg.

2. The antibody of claim 1, wherein said hapten is fentanyl.

3. The antibody of claim 1, wherein said antibody comprises at least one heavy chain CDR, said heavy chain CDR being
(I) a heavy chain CDR1 having an amino acid sequence selected from the group consisting of:
(a) an amino acid sequence as shown in any one of SEQ ID NOs: 32, 33, 34, 35, 36, or 37; and
(b) an amino acid sequence which differs by at least one amino acid exchange, deletion and/or addition from the sequence as shown in any one of SEQ ID NOs: 32, 33, 34, 35, 36, or 37;
(II) a heavy chain CDR2 having an amino acid sequence selected from the group consisting of:
(a) an amino acid sequence as shown in any one of SEQ ID NOs: 53, 54, 56, 57, 58, or 59; and
(b) an amino acid sequence which differs by at least one amino acid exchange, deletion and/or addition from the sequence as shown in any one of SEQ ID NOs: 53, 54, 56, 57, 58, or 59; or
(III) a heavy chain CDR3 having an amino acid sequence selected from the group consisting of:
(a) an amino acid sequence as shown in any one of SEQ ID NOs: 81, 82, 83, 84, 85, 86, 87, 88, 89, or 90; and
(b) an amino acid sequence which differs by at least one amino acid exchange, deletion and/or addition from the sequence as shown in any one of SEQ ID NOs: 81, 82, 83, 84, 85, 86, 87, 88, 89, or 90.

4. The antibody of claim 1, wherein said antibody comprises at least one light chain CDR, said light chain CDR being
(I) a light chain CDR1 having an amino acid sequence selected from the group consisting of:
(a) an amino acid sequence as shown in any one of SEQ ID NOs: 38, 39, 40, or 41; and
(b) an amino acid sequence which differs by at least one amino acid exchange, deletion and/or addition from the sequence as shown in any one of SEQ ID NOs: 38, 39, 40, or 41;
(II) a light chain CDR2 having an amino acid sequence selected from the group consisting of:
(a) an amino acid sequence as shown in any one of SEQ ID NOs: 60, 61, or 62; and
(b) an amino acid sequence which differs by at least one amino acid exchange, deletion and/or addition from the sequence as shown in any one of SEQ ID NOs: 60, 61, or 62; or
(III) a light chain CDR3 having an amino acid sequence selected from the group consisting of:
(a) an amino acid sequence as shown in any one of SEQ ID NOs: 91, 92, 93, 94, 95, 96, or 97; and
(b) an amino acid sequence which differs by at least one amino acid exchange, deletion and/or addition from the sequence as shown in any one of SEQ ID NOs: 91, 92, 93, 94, 95, 96, or 97.

5. The antibody of claim 1, wherein said hapten binding pocket comprises at least the following amino acids:
(i) Trp 47, Tyr 35, Leu 96, Trp 110, Tyr 36, Ile 98, Tyr 91, Thr 106, Asp 108, Tyr 55, Tyr 49, Tyr 101, Gln 89, and Glu 99;
(ii) Val 37, His 35, Tyr 6, Ala 97, Tyr 96, Phe 98, Asp 104, Ile 98, Tyr 55, Tyr 102, Glu 98, Glu 99, Gly 101, Tyr 91, and Tyr 49
(iii) Val 37, Ala 97, His 35, asp 108, Phe 98, Met 98, Tyr 36, Tyr 96, Glu 99, Tyr 55, Tyr 91, Tyr 106, and Gln 89; or
(iv) Trp 110, Phe 98, Ala 97, His 35, Tyr 55, Ile 98, Tyr 96, Tyr 49, Asp 108, Tyr 91, Asn 32, Gln 89, and Glu 99.

6. The antibody of claim 1, wherein said antibody comprises a heavy chain having an amino acid sequence selected from the group consisting of:
(a) an amino acid sequence as shown in any one of SEQ ID NOs: 1, 2, 3, 4, 5, 6, 7, 9 or 10; and
(b) an amino acid sequence which differs by at least one amino acid exchange, deletion and/or addition from the sequence as shown in any one of SEQ ID NOs: 1, 2, 3, 4, 5, 6, 7, 9 or 10.

7. The antibody of claim 1, wherein said antibody comprises a light chain having an amino acid sequence selected from the group consisting of:
(a) an amino acid sequence as shown in any one of SEQ ID NOs: 11, 12, 13, 14, 15, 16, 17, 19, 20 or 21; and
(b) an amino acid sequence which differs by at least one amino acid exchange, deletion and/or addition from the sequence as shown in any one of SEQ ID NOs: 11, 12, 13, 14, 15, 16, 17, 19, 20 or 21.

8. A polynucleotide encoding the antibody of claim 1.

9. The polynucleotide of claim 8, wherein said polynucleotide is RNA or DNA.

10. A vector or expression construct comprising the polynucleotide of claim 8.

11. A host cell comprising the polynucleotide of claim 8.

12. The host cell of claim 11, wherein said host cell is a bacterial cell, a fungal cell, an animal cell or a plant cell.

13. A non-human transgenic organism comprising the polynucleotide of claim 8.

14. The non-human transgenic organism of claim 13, wherein said organism is an animal or a plant.

15. A method of treating and/or preventing a disease or condition in a subject associated with administration of fentanyl or a derivative thereof, the method comprising administering to the subject the antibody as defined in claim 1.
